(19) United States
(12) Patent Application Publication
(10) Pub. No.: US 2005/0118290 A1
(43) Pub. Date: Jun. 2, 2005

(54) Compositions and method for treatment of steroid/nuclear receptor-mediated diseases

(75) Inventors: Eu Leong Yong, Singapore (SG); Toon Yaa Ng, Singapore (SG); Ping Shen, Singapore (SG); Wan Sia Paul Heng, Singapore (SG); Yinhan Gong, Singapore (SG)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: University of Singapore, Singapore (SG)

(21) Appl. No.: 10/727,020

(22) Filed: Dec. 2, 2003

(51) Int. Cl. A61K 35/78

(52) U.S. Cl. 424/757

(57) ABSTRACT

Compositions and method for treatment of steroid/nuclear receptor-mediated diseases. The present invention relates to non-aqueous extracts of Astragalus Membranaceus (HQ) and to compositions thereof. Also provided is a method for the treatment of PPAR-mediated physiological conditions comprising administering to a subject an effective amount of at least one of the above mentioned extracts and also a method for augmenting or synergising the activity of ligands of steroid/nuclear receptors comprising administering to a subject an effective amount of at least one of: an extract of Astragalus Membranaceus (HQ) or a flavonoid compound; in the presence of at least one ligand of steroid/nuclear receptors.

### BACKGROUND OF THE INVENTION

1. Field of the Invention

The invention relates to compositions and methods for the treatment of steroid/nuclear-mediated physiological conditions. In particular, Chinese herb extracts and compounds extracted therefrom are provided.

2. Background Information

Steroid/Nuclear Receptors

Steroid/nuclear receptors are ligand-activated transcription factors that activate genes by binding to the hormone response elements located in their enhancer or promoter regions. Members of the steroid/nuclear receptor superfamily include the androgen receptor (AR), progesterone receptor (PR), glucocorticoid receptor (GR), estrogen receptor (ER) and peroxisome proliferator-activated receptor (PPAR). The steroid/nuclear receptors are commonly organized into domains with specific functions. The N-terminal transactivation domain (TAD) has a ligand-independent activation function whereas; the DNA-binding domain (DBD) enables the receptor to bind to its cognate target site in the promoter of responsive genes. The C-terminal ligand-binding domain (LBD) contains a pocket for specific interactions with small hydrophobic ligands. Upon ligand binding, the steroid/nuclear receptors undergo a conformational change allowing the receptor to interact with DNA, thereby regulating RNA transcription from target genes. Modulation of RNA transcription leads to changes in the level of corresponding proteins, which causes a myriad of physiological activities associated with these receptors.

Peroxisome Proliferator-Activated Receptor (PPAR) and PPAR Activators (Ligands)

PPAR is a member of the nuclear/hormone receptor superfamily, which heterodimerizes with Retinoid X receptor (RXR) and regulates gene expression by binding to Peroxisome Proliferator Responsive Elements (PPRE) in the promoter region of target genes (Mangelsdorf, D. J., Evans, R. M. 1995). There are three known PPAR isotypes, namely, alpha, beta and gamma. PPARγ controls the expression of genes that are involved in the regulation of glucose metabolism, adipogenesis and insulin sensitization and to the processes of carcinogenesis and inflammation (Kersten, S., et al. 2000). Known synthetic ligands of PPARγ include the thiazolidinediones (TZDs) class of compounds, which has been demonstrated clinically to be efficacious in the treatment of Type II diabetes in terms of maintaining plasma glucose, preventing the onset of long-term diabetes complications as well as diabetic resistance (Saltiel, A. R., Olefsky, J. M. 1996). These PPARγ activators have also been developed as anti-proliferative drugs for tumor growth inhibition (Koeffler, H. P. 2003). In addition, the demonstration of their potential anti-inflammatory effects has led to initiation of treatment trials in models of inflammatory diseases and in patients with inflammatory bowel disease (Wada, K., et al. 2001). On the other hand, many studies have demonstrated that PPARα regulates genes involved in lipid metabolism such as fatty acid uptake (fatty acid binding protein (FATP)), β-oxidation (acyl-CoA oxidase) and co-oxidation (cytochrome P450) (Gould Rothberg, B. E., et al. 2001). In line with these observations, the fibrates class of drugs including fenofibrate and gemfibrozil, which are pharmacologic activators of PPARα, lower serum triglycerides (TG) and increase HDL cholesterol in patients with hyperlipidemia. PPARα has also been shown to down-regulate apolipoprotein C_III, a protein which inhibits TG hydrolysis by lipoprotein lipase. This activity of PPARα ligands further contributes to the lipid-lowering effect (Haubenwallner, S., et al. 1995). Moreover, fibrate intervention in cardiovascular disease is likely beneficial because systemic TG reduction could result in less fat accumulation in the heart and at the vessel wall. Therefore, PPARα activators are useful in treating conditions such as dyslipidaemia, atherosclerosis, coronary heart disease, obesity and polycystic ovarian disease.

The many effects of PPAR activators in the regulation of glucose and lipid metabolism have prompted the use of these agents in the treatment of type II diabetes, dyslipidaemia, atherosclerosis, obesity and polycystic ovarian disease. Furthermore, its anti-proliferative effect on breast, prostate and colon cancer has been exploited in the treatment of these cancers. Recently, another therapeutic indication for PPAR activators has been added due to its anti-inflammatory effect to combat inflammatory diseases such as colitis and inflammatory bowel disease.

In view of the vast therapeutic uses of PPAR activators, it would be advantageous to identify compounds that have PPARγ and PPARα agonist or synergistic activity that can be used therapeutically.

Hormones and Hormonal Modulators

Testosterone, estrogens, progesterone and glucocorticoids are steroidal hormones in the body that play a vital role in the human reproductive and immune system by binding to the AR, ER, PR and GR respectively. Testosterone mediates male morphogenesis in utero, gametogenesis and prostate growth at puberty, and the development of prostate cancer in older men. Meanwhile, progesterone together with estrogen, acts on the central nervous system, ovary and uterus to initiate changes in the female reproductive tract that are critical for fertilization of the oocyte, implantation of the embryo and maintenance of pregnancy. On the other hand, glucocorticoids are involved in numerous physiological processes such as endocrine homeostasis, stress responses, lipid metabolism, inflammation and apoptosis. The multiple effects regulated by these hormones on their respective hormonal receptors have provided an opportunity to use synthetic androgens, estrogens, progestins and glucorticoids with selective affinities to these receptors to exert desired effects. However, the use of these agents have been thwarted by the numerous accounts of side effects ranging from increasing risks of cancers, suppression of the hypothalamic-pituitary axis and growth retardation to osteoporosis. The current challenge therefore is to develop agents that maintain their efficacy and are beneficial but have reduced side effects. Thus, it is beneficial to identify chemical compounds or natural products, which augment and synergize the activity of existing steroidal hormones in the body to prevent and decrease side-effects previously experienced with exogenous agents. Such entities are needed for the development of therapeutic compositions and/or nutraceutical applications.

Uses of Extracts and Compounds from Astragalus Membranaceus (HQ)

Astragalus belongs to the Fabaceae (pea) family. Among the 125 different Astragalus species, the dried root of Astragalus Membranaceus is one that is most commonly used medicinally. In China, the dried root of Astragalus Membranaceus is commonly known as Huang qi (HQ), It is used traditionally to reinforce qi and strengthen the superficial resistance, promote the discharge of pus and the growth of new tissue. Classically it is indicated for diabetes mellitus, albuminuria in chronic nephritis, edema due to deficiency of qi, anemia and abscesses (Pharmacopoeia of the People's Republic of China, 1997).

Astragalus Membranaceus (HQ) has been used in combination with other anti-virals or herbs for the treatment of viral diseases (Qian, Z., et al. 1990). It has also beep studied as an adjunct to chemotherapy to decrease the side effects and is a potential anticancer agent (Sun, A., Chiang, C. P. 2001). In addition, some studies have been conducted to determine the usefulness of Astragalus Membranaceus aqueous extract in preventing diabetes (Lu, J., et al. 1999). Many others have studied the use of Astragalus Membranaceus aqueous extract in the treatment of heart diseases (Ma, J., et al. 1998, Yang, Y., et al. 1990). Ethanol extracts of the roots of Astragalus Membranaceus have been described by He Z Q and Wang B Q, 1990. n-butanol extracts of the root of Astragalus Membranaceus are also described in Ma X, et al. 2003. In conclusion, the potential clinical benefits documented to date ranges from viral disease, heart diseases, diabetic and inflammatory syndromes to cancer.

Astragalus Membranaceus root's medicinal properties have been attributed to its polysaccharides which include the Astramembrannins and Astragalosides I to IV (Keiji, K., et al. 1997, Zhang, W. 1997). Similarly, the flavonoids such as afromorsin, calycosin, formononetin and odoratin isolated from this herb could account for the herbs' antioxidant and anti-inflammatory properties (Yoshiaki, S., et al. 1997, Shizuo, T. et al. 1998). Other components isolated include gamma-aminobutyric acid, daucosterol, beta-sitosterol, 4-aminobutanoic acid, dimethyl 4,4′-dimethoxy-5,6:5′,6′-bis(methylenedioxy)biphenyl-2,2′-dicarboxylate palmitic acid, linoleic acid, linolenic acid, folic acid, betaine, praline, asparmide, canavanine and gamma-aminobutyric acid (Zhong Yao Xian Dai Yaq Jiu 1993; Phytochemical Dictionary of the Leguminosae 1994). The concentrations of all these compounds could vary according to cultivation conditions (Ma, X. G. et al. 2002). Ten constituents isolated from ethanol extracts of the roots qi Astragalus Membranaceus have been described by He Z Q and Wang B Q, 1,990. The ten constituents are palmitic acid (I), lupeol (II), β-sitosterol (III), asteragaloside IV (IV), 3S-3-(-) mucronulatol-7-O-β-D-glucopyranoside (V), daucosterol (VI), dimethyl 4,4-dimethoxy-5, 6, 5′, 6′-dimethylenedioxybiphenyl-2,2-dicarboxylate (VII), asparagine (VIII), γ-aminobutyric, acid (IX) and sucrose (X).

Calycosin, one of the major compounds isolated from Astragalus Membranaceus (Lin, L. Z. et al. 2000) can also be isolated from other sources such as Glycyrrhiza palidiflora hairy root cultures (Li, W. et al. 2002). Apart from natural sources, calycosin can also be chemically synthesized (Jain, A. C. et al. 1969). Studies have shown that the antigiardial activity of Machaerium aristulatum is due to the bioactivity of calycosin and formononetin isolated thereof (ElSohly, H. N. et al. 1999). In addition, the antiplasmodial activity of Andira inermis was also accounted for by calycosin (Kraft, C. et al. 2000).

Formononetin, on the other hand, which undergoes microsomal metabolism in the human liver, to calycosin (Tolleson, W. H. et al. 2002) has been better studied. Formononetin has been widely studied for its estrogenic (Miksicek, R. J. 1994, Willard, S. T. and Frawley, L. S. 1998, Kuiper, G. G. J. M. et al. 1998, Morito, K. et al. 2002) and antioxidative capacities (Pool-Zobel, B. L. et al. 2000).

Calycosin and formononetin have also been obtained by n-butanol extracts of the root of Astragalus Membranaceus (Ma, X., et al. 2003, J. Chromatogr., Apr. 11; 992(1-2):193-7).

Calycosin and formononetin belong to the family of isoflavones, which includes genistein among its members. Genistein's properties as a potent phytoestrogen have been widely researched upon. Epidemiological studies have linked the low incidence of prostate cancer among Asian men to their high soy genistein diet. However, the androgenic effects of genistein remain to be elucidated.

Recently, increasing interest has been directed to the use of herbal or natural-source remedies. Additionally, medicinal substances derived from natural products can provide commercial or industrial opportunities for nutraceutical formulations or as food supplements. Finally, compounds identified as the active ingredients in natural products form an important basis for pharmaceutical research.

However, even if the extracts of Astragalus Membranaceus and other herbs and flavonoid compounds isolated therefrom have been investigated, no studies have been reported regarding either the effects of extracts of Astragalus Membranaceus, or of flavonoid compounds isolated therefrom, like calycosin and formononetin, on steroid/nuclear receptors (including PPAR receptors).

### SUMMARY OF THE INVENTION

The present inventors have found that extracts of herb, in particular Astragalus Membranaceus (hereinafter indicated as “HQ”) and/or flavonoid compounds isolated therefrom are useful in the treatment of steroid/nuclear receptors-related physiological conditions.

According to one aspect, the invention provides a hexane, dichloromethane or chloroform extract of Astragalus Membranaceus (HQ), and also to non-aqueous extract of Astragalus Membranaceus (HQ), provided that the HQ extract is not an ethanol or butanol HQ extract. The characteristic of such an extract is that it is enriched in PPAR bioactives.

The invention also provides a composition comprising at least one of the following:
a hexane extract of Astragalus Membranaceus (HQ); a dichloromethane extract of Astragalus Membranaceus (HQ); a chloroform extract of Astragalus Membranaceus (HQ); or a non-aqueous extract of Astragalus Membranaceus (HQ), provided that the HQ extract is not an ethanol or butanol HQ extract.

The composition may further comprise at least one flavonoid compound.

In particular, the invention provides a pharmaceutical or dietary supplement composition for the treatment of steroid/nuclear receptor-mediated physiological conditions comprising an effective amount of at least one extract selected from the group consisting of:
a hexane extract of Astragalus Membranaceus (HQ); a dichloromethane extract of Astragalus Membranaceus (HQ); a chloroform extract of Astragalus Membranaceus (HQ); and a non-aqueous extract of Astragalus Membranaceus (HQ) provided that the HQ extract is not an ethanol or butanol HQ extract.

The pharmaceutical or dietary supplement composition may also be prepared in the form of food product or beverage.

Also provided is a commercial package comprising any form of the above composition and instructions for use.

According to another aspect, the invention provides a method for the treatment of steroid/nuclear (s/n) receptor-mediated physiological conditions comprising administering to a subject an effective amount of at least one extract selected from the group consisting of:
a hexane extract of Astragalus Membranaceus (HQ); a dichloromethane extract of Astragalus Membranaceus (HQ); a chloroform extract of Astragalus Membranaceus (HQ); and a non-aqueous extract of Astragalus Membranaceus (HQ) provided that the HQ extract is not an ethanol or butanol HQ extract.

PPARγ-mediated physiological conditions are diabetes, cancer, polycystic ovarian disease, or inflammatory bowel disease.

PPARα-mediated physiological conditions are dyslipidaemia, atherosclerosis, coronary heart disease or obesity.

According to a further aspect, the inventors have found that flavonoid compounds, for example isoflavonoid compounds isolated from the above mentioned herb extracts, can also be useful for the treatment of PPAR-mediated physiological conditions. Accordingly, the invention also provides a method for the treatment of steroid/nuclear (s/n) receptor-mediated physiological conditions comprising administering to a subject an effective amount of calycosin and/or formononetin, provided that the treatment of cancer of prostate and breast is excluded.

According to another further aspect, the invention provides a method for augmenting or synergizing the activity of ligands of steroid/nuclear receptors comprising administering to a subject an effective amount of at least one of:
i) an extract of Astragalus Membranaceus (HQ);
ii) a flavonoid compound;
iii) a composition according to any embodiment of the invention; in the presence of at least one ligand of steroid/nuclear receptors.

The steroid/nuclear receptor ligand may be an anti-diabetic or hypolipidemic drug. The ligand can be a hormone, for example androgen, progestogen or glucocorticoids.

In particular, in the method above for augmenting or synergizing the activity of ligands of steroid/nuclear receptors:
a) the ligand is androgen and the subject is or is not under disease or condition of male infertility, chronic bone and muscle mass loss, geriatric andropause, androgen insensitivity syndromes, Klinefelter syndrome or cryptorchidism;
b) the ligand is progestogen and the subject is or is not under disease or condition of postmenopausal hormone replacement therapy, female infertility, endometrial cancer, secondary amenorrhea, functional uterine bleeding or menstrual disorders; and/or
c) the ligand is glucocorticoid and the subject is or is not under disease or condition of autoimmune diseases, arthritis, post-operative graft rejection or asthma.

In the method above, flavonoid compounds can also be used. For example, suitable flavonoid compounds are calycosin, formononetin, genistein, afromorsin, biochanin A, coumestrol, odoratin, daidzein and the like.

In particular, the method for augmenting or synergizing the activity of ligands of steroid/nuclear receptors is a method for the treatment of PPARγ-mediated conditions selected from the group consisting of: diabetes, atherosclerosis, polycystic ovarian disease, hormone-dependent cancer of the breast, colon or prostate, or inflammatory bowel disease.

According to another aspect, the invention provides a method for preparing an extract of Astragalus Membranaceus, comprising treating Astragalus Membranaceus plant or part of the plant with hexane, dichloromethane and/or chloroform.

According to another further aspect, the invention provides a method for screening and/or discovering compounds or extracts capable of augmenting or synergizing the activity of ligands of steroid/nuclear receptors comprising;
contacting or mixing a compound or extract to a composition comprising at least one ligand of steroid/nuclear receptors, and determining the augment or synergism of the activity of the ligand.

In particular the ligand is bound to a pocket of the steroid/nuclear receptors and the compound or extract does not bind specifically to the ligand-binding pocket of the steroid/nuclear receptor.

The ligand may be an endogenous androgen, progestogen, glucocorticoid and/or a PPAR agonist.

### BRIEF DESCRIPTION OF THE DRAWINGS

FIGS. 1A and 1B: Dose response effect of HQ extract on PPARα and PPARγ activity. HeLa cells expressing chimeric GAL₄₋DNA-binding domain and the LBDs of (A) PPARγ and (B) PPARα were exposed to increasing doses of an ethanolic extract of HQ. Ligands that bind to these chimeric Ga14_{DBD}-PPAR_{LBD} receptors result in the activation of a co-transfected reporter gene, comprising five copies of UASg cloned upstream of a Luciferase gene. PPAR bioactivity was measured and compared to the reference PPARγ ligands 15-deoxy-δ12,14, Prostaglandin J2 (15dPGJ2) and Pioglitazone in (A), and to the reference PPARα ligand WY14643 in (B). PPAR activity of the test materials are expressed as fold increase in luciferase activity compared to cells exposed to vehicle only. Each data point is the mean±SEM.

FIGS. 2A and 2B: Synergistic effect of genistein on androgen action. HeLa cells expressing the human androgen receptor, and a luciferase reporter gene driven by androgen response elements were exposed to increasing doses of (A) genistein in the presence, or absence, of a fixed concentration of DHT (10×10⁻⁹M); or (B) DHT in the presence, or absence, of a fixed concentration (3×10⁻⁶ M) of genistein. AR activity of the test materials is expressed as fold increase in luciferase activity compared to cells exposed to vehicle only. Each data point is the mean±SEM.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The present inventors have screened traditional Chinese herbs, that is, herbs traditionally indicated for diabetes mellitus or “thirst-disease” in order to identify activators of steroid/nuclear receptors, in particular PPAR, more in particular the PPARγ and PPARα, using a cell-based reported gene system. From this screening, Astragalus Membranaceus (also known as “Huang Qi”) non-aqueous extracts were found to exhibit novel steroid/nuclear receptors, in particular PPARγ and PPARα, agonist activity.

Accordingly, the invention provides non-aqueous extracts of Astragalus Membranaceus (HQ), provided that the HQ extract is not an ethanol or butanol HQ extract. In particular, the invention is addressed to a hexane, dichloromethane or chloroform extract of Astragalus Membranaceus (HQ).

These extracts have proved to be enriched in steroid/nuclear (s/n) receptor bioactives, in particular, in PPARγ and PPARα bioactives.

Being strong activators of the steroid/nuclear (s/n) receptor family, and in particular of the peroxisome proliferator-activated receptor (PPAR) family of receptors, the above mentioned Astragalus Membranaceus extract may therefore be useful for the treatment of conditions mediated through s/n receptor family such as diabetes, dyslipidaemia, atherosclerosis, obesity, polycystic ovarian disease, hormone-dependent cancers of the breast, colon and prostate and inflammatory diseases.

“Steroid/nuclear receptor” refers to ligand-activated transcription factors that activate genes by binding to the hormone response elements located in their enhancer or promoter regions. Members of the steroid/nuclear receptor superfamily include the androgen receptor (AR), progesterone receptor (PR), glucocorticoid receptor (GR), estrogen receptor (ER) and peroxisome proliferator-activated receptor (PPAR). PPAR receptors include PPARγ and PPARα.

The invention also relates to novel methods to measure and standardize the bioactivity of Astragalus Membranaceus and its extracts based on the s/n receptor, in particular on the PPAR, activity.

The extracts according to the invention can be prepared from plant or parts of plants of Astragalus Membranaceus, in particular, from the roots of the plants. The extracts can be prepared according to any standard method known in the art, for example those described by He, Z. Q. and Wang, B. Q. 1990 and by Ma, X. et al. 2003. In the method of the present invention, dried roots of the plants are preferably milled into small pieces and then extracted with a non-aqueous solvent, preferably with hexane, dichloromethane and/or chloroform. Methods for preparation of the extracts according to the invention are disclosed in the examples.

Reference to the Astragalus Membranaceus herb also encompasses natural and artificial created variants of Astragalus Membranaceus. An artificially created variant includes a variant made by selective breeding or by genetic manipulation. A part of Astragalus Membranaceus includes the bark, leaf, stem, root, flower, seed or other reproductive or vegetative portion of the plant or a combination of two or more of these portions.

The inventors have found that the extracts according to the invention are enriched with flavonoid compounds. In particular, isoflavonoid compounds like calycosin and formononetin.

The inventors have found that calycosin is a potent steroid/nuclear receptors agonist, in particular a potent PPARγ agonist. Calycosin may therefore be useful for the treatment of conditions mediated through PPARγ such as diabetes, atherosclerosis, polycystic ovarian diseases, hormone-dependent cancers of the breast, colon and prostate, and inflammatory bowel diseases.

The inventors have also found that formononetin is a potent steroid/nuclear receptors agonist, in particular a potent PPARα and PPARγ agonist. Formononetin may therefore be useful for the treatment of conditions mediated through both PPARγ and PPARα such as diabetes, dyslipidaemia, atherosclerosis, obesity, polycystic ovarian disease, hormone-dependent cancers of the breast, colon and prostate, and inflammatory bowel diseases. Other flavonoid compounds suitable as agonists of steroid/nuclear receptors are also within the scope of the present invention.

Calycosin, formononetin and other flavonoid compounds can be prepared according to any standard method known in the art. Methods of preparation of calycosin and formononetin are disclosed in the examples. Calycosin, formononetin and other suitable flavonoid compounds can also be chemically synthesized according to standard methods.

The term “agonist,” as used herein, refers to at least one extract or flavonoid compounds according to the invention, a mix thereof, or a composition comprising the extract(s) or flavonoid compound(s) according to the invention which, when bound to any steroid/nuclear receptor, activates the steroid/nuclear receptor. The term “modulate,” as used herein, refers to a change or an alteration in the biological activity of any steroid/nuclear receptor. Modulation may be an increase or a decrease in protein activity, a change in binding characteristics, or any other change in the biological, functional or immunological properties of the steroid/nuclear receptor.

The present invention also relates to composition or formulation comprising at least one extract and/or at least one flavonoid compound according to the invention.

The composition according to the invention comprises a part of Astragalus Membranaceus or a botanical or horticultural equivalent thereof or an extract thereof or chemical or functional equivalents of the extract or a purified or chemical synthetic form of one or more components of the extract wherein said composition is effective in modulating a steroid/nuclear receptors-mediated condition in a subject. The composition of the present invention may also be referred to as a herbal composition, natural medicine, a formulation and/or a formulation or composition with medicinal or ameliorating properties. The terms “formulation” and “composition” are herein used interchangeably.

The term composition includes an extract of Astragalus Membranaceus or parts thereof in liquid, solid or aerosol or vapor form. In particular, the formulation comprises a non-aqueous extract of Astragalus Membranaceus. More in particular, the extract is not an ethanolic and butanolic extract. Preferably, the extract is a hexane, dichloromethane or chloroform extract. The formulation may also comprise a flavonoid compound which is deemed suitable by any skilled person in the art. For example, calycosin, formononetin genistein, afromorsin, biochanin A, coumestrol, odoratin, daidzein and the like. However, the list of suitable flavonoid compounds is not limited to these compounds. Any suitable flavonoids, for example any suitable flavonoids described in a public or private flavonoid or isoflavonoid database (see below) is within the scope of the present invention.

The composition may therefore comprise of at least one of the following: a hexane extract of Astragalus Membranaceus (HQ); a dichloromethane extract of Astragalus Membranaceus (HQ); a chloroform extract of Astragalus Membranaceus (HQ); a non-aqueous extract of Astragalus Membranaceus (HQ), provided that the HQ extract is not an ethanol or butanol HQ extract; or a flavonoid compound.

In particular, the invention relates to a pharmaceutical or dietary supplement composition for the treatment of steroid/nuclear (s/n) receptor-mediated physiological conditions comprising of an effective amount of at least one extract selected from the group consisting of: a hexane extract of Astragalus Membranaceus (HQ); a dichloromethane extract of Astragalus Membranaceus (HQ); a chloroform extract of Astragalus Membranaceus (HQ); and a non-aqueous extract of Astragalus Membranaceus (HQ) provided that the HQ extract is not an ethanol or butanol HQ extract; and further at least one flavonoid compound.

In particular, s/n receptor-mediated physiological condition is a PPAR-mediated disease and is diabetes, cancer, polycystic ovarian disease, inflammatory bowel disease, dyslipidaemia, atherosclerosis, coronary heart disease or obesity.

The subject composition or formulation in the form of Astragalus Membranaceus extract may be administered in any suitable form including ingestion, topical application or via vapor or aerosol means. The term “ingestion” includes administering the herb or extract via edible or liquid means.

For in vivo applications, the extract or plant parts can be incorporated into a pharmaceutically acceptable formulation including a carrier or diluent for administration. Those skilled in the art will readily determine suitable dosage levels. Exemplary pharmaceutically acceptable carriers include carriers suitable for oral, intravenous, subcutaneous, intramuscular, and the like administration. Administration in the form of creams, lotions, tablets, dispersible powders, granules, syrups, elixirs, sterile aqueous or non-aqueous solutions, suspensions or emulsions, and the like, are contemplated. For the preparation of oral liquids, suitable carriers include emulsions, solutions, suspensions, syrups and the like, optionally containing additives such as wetting agents, emulsifying and suspending agents, sweetening, flavoring and perfuming agents, and the like. For the preparation of fluids for parenteral administration, suitable carriers include sterile aqueous or non-aqueous solutions, suspensions or emulsions. Examples of non-aqueous solvents or vehicles are propylene glycol, polyethylene glycol, vegetable oils, such as olive oil and corn oil, gelatin, and injectable organic esters such as ethyl oleate. Such forms of dosage may also contain additional ingredients such as preserving, wetting, emulsifying and dispersing agents. Formulations may be sterilized, for example, by filtration through a bacteria-retaining filter, by incorporating sterilizing agents into the compositions, by irradiating the compositions, or by heating the compositions. They can also be manufactured as solutions in sterile water, or in some other sterile injectable medium, immediately before use.

“Suitable dosage levels” include reference to levels sufficient to provide circulating concentrations high enough to effect activation of steroid/nuclear receptor(s).

The composition according to the invention may comprise of an effective amount of 1 to 1000 μg/mL (1 to 1000×10⁻⁶ g/mL) of extract (in plasma) for the treatment of s/n receptor-mediated physiological conditions. In particular, the composition may comprise of an effective amount of 1 to 1000 μg/mL of extract (in plasma) for the treatment of PPARα and/or PPARγ-mediated physiological conditions.

The invention also provides a food product and/or beverage comprising of the composition, pharmaceutical composition or dietary supplement composition of the invention.

The invention further provides a commercial package comprising of the any composition according to the invention.

According to another aspect, the invention provides a method for the treatment of steroid/nuclear (s/n) receptor-mediated physiological conditions comprising administering to a subject an effective amount of at least one extract selected from the group consisting of: a hexane extract of Astragalus Membranaceus (HQ); a dichloromethane extract of Astragalus Membranaceus (HQ); a chloroform extract of Astragalus Membranaceus (HQ); and a non-aqueous extract of Astragalus Membranaceus (HQ), provided that the HQ extract is not an ethanol or butanol HQ extract.

As indicated in the example, considering 100% absorption in an adult human with a normal blood volume of 5 L, doses of about 5 to 5000 mg (5 to 5000×10⁻³ g) of dried extract are required for a clinical effect.

Accordingly, the method comprises treating the subject with about 1 to 1000 μg of extract per mL of blood volume.

More in particular, the invention provides a method comprising treating the subject with 3 to 5000 mg of dried extract for the treatment of PPARα- and/or PPARγ-mediated physiological conditions. Further, the invention provides a method comprising treating the subject with 1 to 1000 μg of extract per mL of blood volume for the treatment of PPARα- and/or PPARγ-mediated physiological conditions.

More particular values and doses of extracts of the invention and flavonoid compounds are disclosed in the examples.

In particular, in the method of the invention, the s/n receptor-mediated physiological condition is a PPARγ-mediated physiological condition and is diabetes, cancer, polycystic ovarian disease, or inflammatory bowel disease or is a PPARα-mediated physiological condition and is dyslipidaemia, atherosclerosis, coronary heart disease or obesity.

The invention further relates to a method for the treatment of steroid/nuclear (s/n) receptor-mediated physiological conditions comprising administering to a subject an effective amount of calycosin and/or formononetin, provided that the treatment of cancer of prostate and breast is excluded.

In particular, the s/n receptor-mediated physiological condition is a PPAR-mediated physiological condition and is diabetes, atherosclerosis, inflammatory bowel disease, polycystic ovarian disease, obesity, dyslipidaemia and/or the cancer of colon.

The present inventors have further found, and it represents a further object of the present invention, Astragalus Membranaceus extract has a synergistic activity on known ligands of s/n receptors, in particular, on known PPAR ligands. Accordingly, Astragalus Membranaceus extract can be used as nutraceutical agents to augment the effects of standard drugs, for example anti-diabetic and hypolipidemic drugs.

As used herein, “synergistic activity” or the equivalent “synergistic effect” refers to any effect of two chemicals acting together which is greater than the simple sum of their effects when acting alone: such chemicals are said to show synergism. More in particular, a first chemical is at least one Astragalus Membranaceus extract, flavonoid compound, or mixer thereof, or a composition comprising the Astragalus Membranaceus extract(s) or flavonoid compound(s) of the invention. The second chemical is any known ligand of s/n receptors.

The inventors have also found that isoflavonoid compounds isolated from extracts of Astragalus Membranaceus augment and synergize the activity of ligands of s/n receptors. In particular, the activity of hormones acting through the PPAR, AR, PR, and GR. Those skilled in the art will realize that this novel augmentative ability of extracts from Astragalus Membranaceus can be utilized to boost the effects of endogenous or exogenous PPARα and PPARγ ligands, androgens, progestogens and glucocorticoids in diseases or conditions due to the deficiency of these hormones. Examples of conditions requiring an augmentation of activity by androgens are male infertility, chronic bone and muscle mass loss, geriatric andropause, androgen insensitivity syndromes, Klinefelter syndrome and cryptorchidism; by progestogens are postmenopausal hormone replacement therapy, female infertility, endometrial cancer, secondary amenorrhea, functional uterine bleeding as well as related menstrual disorders (caused by hormonal deficiency or imbalance); by glucocorticoids are autoimmune diseases, arthritis, post-operative graft rejection and asthma. It can also be used to augment the effects of these hormones in normal people where such boosting effects are desired.

The invention is however not limited to flavonoid compounds isolated from plant, part of plant or extracts of Astragalus Membranaceus, but encompasses any suitable flavonoid compound isolated from different sources or chemically synthesized.

In particular, calycosin can augment the activity of pioglitazone, WY14643, dihydrotestosterone, progesterone and estradiol on the PPARγ, PPARα, AR, PR and ERα signaling systems. Those skilled in the art will realize that this novel augmentative ability of calycosin can be utilized to boost the effects of endogenous or exogenous PPARα and PPARγ ligands, androgens, estrogens, and progestogens in diseases due to the deficiency of these hormones. Examples of such diseases or conditions requiring an augmentation of activity by androgens are male infertility, chronic bone and muscle mass loss, geriatric andropause, androgen insensitivity syndromes, Klinefelter syndrome and cryptorchidism; by progestogens and estrogens are postmenopausal hormone replacement therapy, female infertility, endometrial cancer, secondary amenorrhea, functional uterine bleeding as well as related menstrual disorders (caused by hormonal deficiency or imbalance). It can also be used to augment the effects of these hormones in normal people where such boosting effects are desired.

Formononetin can augment the effects of WY14643, dihydrotestosterone, progesterone and Estradiol on the PPARα, AR, PR and ERα signaling systems. Those skilled in the art will realize that this novel augmentative ability of formononetin can be utilized to boost the effects of endogenous or exogenous androgens, estrogens, progestogens and PPARα ligands in diseases due to a deficiency of these hormones. Examples of such diseases requiring an augmentation of activity by androgens are male infertility, chronic bone and muscle mass loss, geriatric andropause, androgen insensitivity syndromes, Klinefelter syndrome and cryptorchidism; by progestogens are postmenopausal hormone replacement therapy, female infertility, endometrial cancer, secondary amenorrhea, functional uterine bleeding as well as related menstrual disorders (caused by hormonal deficiency or imbalance). It can also be used to augment the effects of these hormones in normal people where such boosting effects are desired.

The inventors have also found that that flavonoids other than calycosin and formononetin, such as genistein, daidzein, and the like, have the novel ability to augment the activity of pioglitazone, WY14643, dihydrotestosterone, progesterone and estradiol on the PPARγ, PPARα, AR, PR and ERα signaling systems. Those skilled in the art will realize that this novel augmentative ability of flavonoids can be utilized to boost the effects of endogenous or exogenous PPARα and PPARγ bands, androgens, estrogens, and progestogens in diseases or conditions due to a deficiency of these hormones. Examples of such diseases and conditions requiring an augmentation of activity by androgens are male infertility, chronic bone and muscle mass loss, geriatric andropause, androgen insensitivity syndromes, Klinefelter syndrome and cryptorchidism; by progestogens and estrogens are postmenopausal hormone replacement therapy, female infertility, endometrial cancer, secondary amenorrhea, functional uterine bleeding as well as related menstrual disorders (caused by hormonal deficiency or imbalance). It can also be used to augment the effects of these hormones in normal people where such boosting effects are desired.

Accordingly, the invention relates to a method for augmenting or synergizing the activity of ligands of steroid/nuclear receptors comprising contacting at least one of: i) an extract of Astragalus Membranaceus (HQ); ii) a flavonoid compound; or; iii) a composition according to any embodiment of the invention; in the presence of at least one ligand of steroid/nuclear receptors.

According to one embodiment, the invention relates to a method for augmenting or synergizing the activity of ligands of steroid/nuclear receptors comprising administering to a subject an effective amount of at least one of: i) an extract of Astragalus Membranaceus (HQ); ii) a flavonoid compound; or iii) a composition according to any embodiment of the invention; in the presence of at least one ligand of steroid/nuclear receptors.

In particular, in the method of the invention the ligand is a PPAR ligand. The ligand is for example an anti-diabetic or hypolipidemic drug, for example, a hormone. The hormone can be an androgen, progestogen, estrogen or glucocorticoid.

In particular, the invention relates to a method wherein: a) the ligand is androgen and the subject is or is not under disease or condition of male infertility, chronic bone and muscle mass loss, geriatric andropause, androgen insensitivity syndromes, Klinefelter syndrome or cryptorchidism; b) the ligand is progestogen or estrogen and the subject is or is not under disease or condition of postmenopausal hormone replacement therapy, female infertility, endometrial cancer, secondary amenorrhea, functional uterine bleeding or menstrual disorders; and/or c) the ligand is glucocorticoid and the subject is or is not under disease or condition of autoimmune diseases, arthritis, post-operative graft rejection or asthma.

Any suitable known or not yet discovered flavonoid compound, and in particular isoflavonoid compound, is within the scope of the present invention. A number of flavonoids, and in particular isoflavonoids, is for example described in USDA-Iowa State University Database on the Isoflavone Content of Foods, Release 1.3-2002, and in USDA Database for the Flavonoid Content of Selected Foods—2003 http://www.nal.usda. gov/fnic/foodcomp/Data/isoflav/isoflav.html) and (http://www.nal.usda.gov/fnic/foodcomp/Data/Flav/flav.html) (both of them herewith incorporated by reference). It will be evident to any skilled person how to choose the suitable flavonoid compound for the purpose of the present invention. For example, flavonoid compounds for the purpose of the present invention may be (but are not limited to) calycosin, formononetin, genistein, afromorsin, biochanin A, coumestrol, odoratin, daidzein and the like.

More in particular, the method is for the treatment of PPARγ-mediated conditions selected from the group consisting of: diabetes, atherosclerosis, polycystic ovarian disease, hormone-dependent cancer of the breast, colon or prostate, or inflammatory bowel disease.

The invention also relates to new method(s) to augment the action of steroid/nuclear receptors such as AR, PR GR, ER and PPAR that do not involve specific ligand binding to the ligand-binding pocket of steroid receptors. This finding allows the development of new drug discovery screening platforms to search for compounds that may augment the action of liganded-steroid receptors.

Accordingly, the invention provides a method for screening and/or discovering compounds or extracts capable of augmenting or synergizing the activity of ligands of steroid/nuclear receptors comprising; contacting (or mixing with) a compound or extract to a composition comprising at least one ligand of steroid/nuclear receptors, and determining the augment or synergism of the activity of the ligand. In particular, the ligand is bound to a pocket of the steroid/nuclear receptors and the compound or extract does not bind specifically to the ligand-binding pocket of the steroid/nuclear receptor. More in particular, the ligand is an endogenous androgen, progestogen, glucocorticoid and/or a PPAR agonist. Such a screening and/or discovery method is carried out according to standard methods known to those skilled in the art.

The present invention also enables the design of synthetic or natural compounds related to genistein, daidzein, calycosin, or formononetin and the like that have the above agonistic and synergistic effects.

### EXAMPLES

The present invention is not limited to the embodiments described. Many variations are possible within the scope of the invention as will be clear to a skilled reader. In an effort to discover and characterize herbs with PPAR activity, a reporter gene assay system (as provided in detail below) driven by a chimeric PPAR receptor was used.

### Example 1

### Chimeric PPARα and PPARγ Receptors for Detecting PPAR Activators

In an example of such a reporter gene assay, a plasmid containing chimeric Ga14_{DBD-}PPAR_{LBD} receptor cDNA was constructed by excising the ligand binding domain (LBD) of full length PPARγ and PPARα and ligating the resulting fragments in frame to the Ga14-DNA binding domain (DBD) of Saccharomyces cerevisiae in a pM vector (Clontech). Specifically, to construct Ga14_{DBD-}PPARγ_{LBD} plasmid, pSG5 expression vector (Stratagene) containing full length PPARγ was excised with Rsa I and blunt ligated to Hind III site of pMGa14_{DBD} expression plasmid (Clontech). To construct Ga14_{DBD-}PPARα_{LBD} plasmid, pSG5 expression vector (Stratagene) containing full length PPARα was excised with BstU I and BamH I and blunt-end ligated in frame to BamH I site of pMGa14_{DBD} expression plasmid (Clontech). The resulting plasmids, when expressed in HeLa cells, resulted in the production of corresponding Ga14_{DBD-}PPARγ_{LBD} proteins. Ligands that bind to these chimeric PPAR receptors cause the activation of a co-transfected reporter gene, comprising five copies of upstream activating sequences for galactose (UASg) cloned in tandem to a Luciferase gene (Olsson, O. et al. 1988). Ga14 _{DBD} binds very strongly to the exogenous UASg promoter, thereby giving rise to a sensitive and specific assay. To measure PPARγ and PPARα agonist activity, HeLa cells were grown in 24-well microtiter plates and then transiently co-transfected with the two plasmids (chimeric PPAR receptor and UASg reporter gene) using Lipofectamine (Invitrogen). Cells were exposed to herbal extracts and pure compounds in RPMI 1640 medium, supplemented with 10% charcoal-treated fetal calf serum, 2 mM L-glutamine, 0.1 mM non-essential amino acids and 1 mM sodium pyruvate for 40 hours at 37° C. in a 5% carbon dioxide incubator. Concurrently, cells exposed to known PPAR ligands served as positive controls. Replicate wells, exposed to the vehicle solvent (i.e., ethanol or methanol) in which herbal extracts were dissolved, were used as negative controls. After the 40 hr incubation period, cells were rinsed with phosphate buffered saline (PBS), lysis buffer was added, and cell lysates were collected for measurement of luciferase activity using a luminometer. PPAR activity of the herbal extracts/pure compounds was expressed as fold-increase in luciferase activity as compared to that observed in negative control cells. All data points are in triplicate. The data points represent the mean of triplicate wells. Luciferase activity in such an assay accurately reflects the activity of any ligands on PPARγ or PPARα.

### Example 2

### Screening and Discovery of Herbal Extracts that Stimulate PPAR Activity

Traditional Chinese medical texts and pharmacopoeia were reviewed. Raw herbal material with purported actions against “thirst” or described to “invigorate qi were purchased from commercial retailers. These herbs are listed in Table 1. Herbs were milled and then macerated with ethanol for 3 days at 37° C., after which the menstruum was filtered with filter paper (11 μm pore size). Filtered extracts were dried in a rotary evaporator. Dried extracts were weighed and re-suspended in 100% ethanol to a concentration of 250×10⁻⁻³ g/ml. Each herbal extract was screened for PPARα and PPARγ activity in vitro at a final concentration of 250×10⁻⁶ g/ml, using the bioassay described in Example 1. From the preliminary screening, three herbal extracts (Astragalus Membranaceus, Trichosantes spp. and Atractylis orata) were found to exhibit PPAR stimulatory activity. Very strong activity was observed with Astragalus Membranaceus, with the extract stimulating PPARγ and PPARα activity 19- and 4-fold higher respectively, compared to controls exposed to vehicle (i.e., ethanol or methanol) only (Table 1). Moderate, but significant, PPAR-stimulatory activity was observed with Trichosantes spp. and Atractylis orata extracts.

TABLE 1
Screening of herbs for PPAR activity using chimeric
Gal4_{DBD-}PPAR_{LBD} receptor and five copies of UASg
cloned upstream of a Luciferase reporter gene in HeLa cells

| | PPARγ | PPARα |
|---|---|---|
| | activity (fold | activity (fold |
| Herbal extract | increase over | increase over |
| (250 × 10⁻⁶ g/ml) | vehicle ± SE) | vehicle ± SE) |
| Astragalus membranaceus | 19.6 ± 0.2 | 4.4 ± 0.1 |
| Trichosantes spp. | 3.0 ± 0.1 | 1.4 ± 0.0 |
| Atractylis orata | 2.9 ± 0.2 | 0.5 ± 0.0 |
| Anemarrhena asphodeloides | 1.9 ± 0.1 | 0.4 ± 0.0 |
| Scrophularia ningpoensis | 1.3 ± 0.1 | 0.8 ± 0.0 |
| Lycium chinense | 0.9 ± 0.1 | 0.5 ± 0.1 |
| Schisandra spp. | 1.8 ± 0.1 | 0.6 ± 0.0 |

### Example 3

### Astragalus Membranaceus (HQ) Extract Displays PPARγ and PPARα Agonist Activity

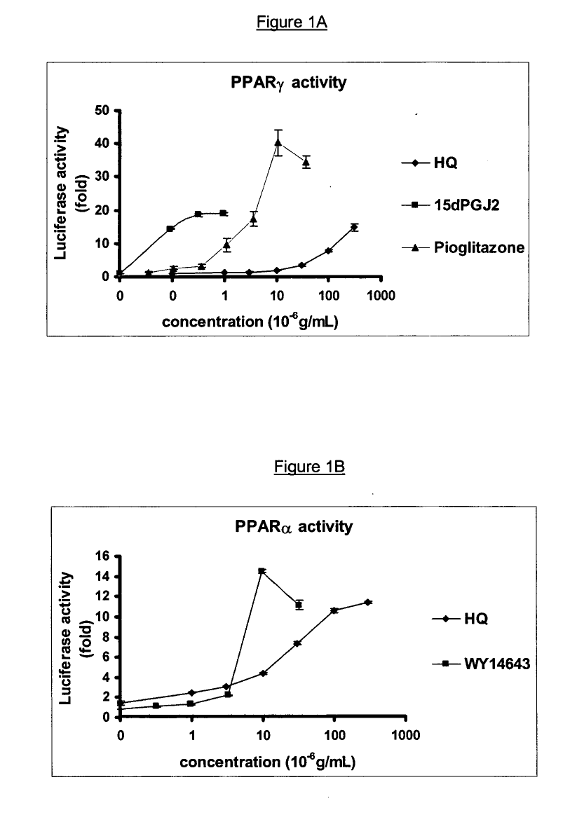

To quantify the activity of this Astragalus Membranaceus (HQ) extract against reference compounds, a 100% ethanolic extract of HQ was compared to the physiological PPAR ligand, 15-deoxy-612,14, Prostaglandin J2; the antidiabetic PPARγ compound, Pioglitazone; and the PPARα ligand, WY14643 in PPAR bioassays. As can be seen in FIG. 1(A), PPARγ activity of HQ was first observed at a dose 30×10⁻⁶ g/mL, rising to a peak at 300×10₋₆ g/mL. The activity of HQ at its peak was equivalent to the maximum activity observed with 15DPGJ2, and was half of that observed with pioglitazone. The EC₅₀ for HQ, 15DPGJ2 and pioglitazone were 100, 0.5 and 5×10⁻⁶ g/mL, respectively, indicating that the ethanolic extract of HQ was only about 20 times less potent than pioglitazone, a pure compound currently used for treatment of diabetes mellitus. The bioassay system given in Example 1 was repeated with COS-7 cells in place of HeLa cells and similar results were obtained. Similar dose responses for HQ was also obtained when transient co-transfection of the full-length human PPARγ receptor and PPAR response element (PPRE) cloned upstream of a luciferase reporter gene in HeLa cells. Thus HQ ethanolic extract is an activator of PPARγ and therefore can be used in the treatment of PPARγ-mediated conditions. Based on FIG. 1A, doses of HQ ethanolic extract resulting in 30 to 1000×10⁻⁶ g/mL of HQ in plasma are predicted to have a therapeutic effect. Assuming 100% absorption in an adult human with a normal blood volume of 5 L, doses of 150 to 5000×10⁻³ g of HQ ethanolic extract would be required for a clinical effect. Thus HQ can be used to treat conditions mediated by PPARγ like diabetes, polycystic ovarian disease, cancer, and inflammatory bowel disease.

HQ ethanolid extract was also a ligand and activator for PPARγ. As can be seen in FIG. 1B, PPARα activity was first observed at a dose 1×10⁻⁶ g/mL, rising to a peak at 300×10⁻⁶ g/mL. The PPARα activity of HQ at its peak was equivalent to maximal activity observed with the pure agonist, WY14643. Thus although the dose response curve of HQ was shifted to the left compared to WY14643, the EC₅₀ for HQ and WY14643 were comparable being 20×10⁶ g/mL and 4×10⁻⁶ g/mL, respectively. Thus HQ ethanolic extract is also a bona-fide activator of PPARα and therefore can be used in the treatment of PPARα-mediated conditions. Based on FIG. 1B, effective doses of HQ should result in 1 to 1000×10⁻⁶ g/mL of HQ in plasma to observe a therapeutic effect. Assuming 100% absorption in an adult human with a normal blood volume of 5 L, doses of 5 to 5000×10⁻³ g of HQ would be required for a therapeutic effect. Thus HQ can be used to treat conditions mediated by PPARα like dyslipidaemia, atherosclerosis, coronary heart disease and obesity.

### Example 4

### Synergistic Action of HQ Extract on the Activity of Known PPAR Ligands

HQ extract was prepared with ethanol and assayed as described in Example 1. Cells were exposed to increasing concentrations of HQ extract in the presence of fixed concentrations of the reference PPAR ligand, ciglitazone (Table 2). Maximal PPARγ active was observed at 3×10⁻⁶ g/mL or 10×10⁻⁶ M of ciglitazone. In comparison, HQ extract was able to stimulate PPARγ activity maximally at a dose of 300×10⁻⁶ g/mL. HQ was also able to dose dependently increase PPARγ activity of saturating doses of ciglitazone. The PPARγ activity of ciglitazone and HQ was 450% higher than that observed with ciglitazone alone at saturating doses. This indicates that HQ extract contains compounds that act directly or indirectly on sites other than the ligand-binding pocket of the LBD. This ability of HQ extract to increase the activity of PPARγ drugs can be used to boost the effects of these drugs, thereby increasing their effectiveness and allowing lower and less toxic dosages to be used.

TABLE 2
PPARγ activity of ciglitazone, HQ and ciglitazone/HQ combination.
Data is expressed as a percentage of maximal ciglitazone activity

| | Luciferase activity ± SEM | | |
|---|---|---|---|
| | (% ciglitazone 10 × 10⁻⁶ M) | | |
| Concentration | Ciglitazone | HQ | HQ extract + ciglitazone |
| (×10⁻⁶ g/mL) | alone | extract alone | 10 × 10⁻⁶ M |
| 0.0 | 8.54 ± 1.08 | 6.72 ± 0.11 | 100 ± 2.91 |
| 0.1 | 15.4 ± 0.81 | ND | ND |
| 0.3 | 21.7 ± 1.52 | ND | ND |
| 1.0 | 55.3 ± 7.99 | 8.83 ± 0.54 | 107 ± 9.88 |
| 3.0 | 100 ± 6.13 | 9.23 ± 0.87 | 139 ± 2.81 |

| | Luciferase activity ± SEM | | |
|---|---|---|---|
| | (% ciglitazone 10 × 10⁻⁶ M) | | |
| Concentration | Ciglitazone | Concentration | Ciglitazone |
| (×10⁻⁶ g/mL) | alone | (×10⁻⁶ g/mL) | alone |
| 10.0 | ND | 12.2 ± 0.67 | 168 ± 8.13 |
| 30.0 | ND | 23.0 ± 1.19 | 172 ± 2.45 |
| 100.0 | ND | 52.1 ± 3.03 | 236 ± 3.70 |
| 300.0 | ND | 99.4 ± 6.69 | 442 ± 30.7 |

ND: Not Determined

### Example 5

### Agonist Effect of HQ on ERα and ERβ Reporter-Gene Systems

The capacity of HQ extract to activate other steroid receptors, namely AR, PR, GR, ERα and ERβ was examined. Plasmids encoding either AR (Brinkmann, A. O. et al., 1989), PR (Vegeto, E., 1992) and GR (Muller, M., et al., 1991) were co-expressed with a reporter gene, (ARE)₂-Luc, that contains hormone-response elements that are common to all three receptors. The (ARE)₂-Luc plasmid consist of two repeats of Androgen Response Element (ARE) cloned upstream of the Luciferase Reporter gene in a pGL3-Basic vector (Promega).

ERα and ERβ activity were assayed using plasmids encoding either ERα or ERβ co-transfected with the ERE-LUC reporter gene. To assay estrogen receptor agonist activity, HeLa cells were transiently co-transfected with two plasmids (Tcherepanova, et al., 2000) using Lipofectamine. The first plasmid comprised DNA encoding human estrogen receptor (either ERα or ERβ), and the second plasmid comprised an estrogen-driven reporter system comprising: a luciferase reporter gene (LUC) whose transcription is under the control of upstream regulatory elements comprising 4 copies of the vitellogenin estrogen response element (ERE) cloned into the Mouse Mammary Tumor Virus (MMTV) promoter (the full name of the reporter system being ‘MMTV-ERE-LUC’).

AR, PR, GR and ERα and ERα activities were determined by comparing luciferase activity in test cells with control cells exposed to vehicle (i.e., ethanol or methanol) only, analogous to the determination of PPAR activity as described in the previous examples. HQ extract displayed strong ERP activity, resulting in a maximal 68-fold increase (comparable to 200% maximal activity of estradiol) in ERα activity at a dose of 300×10⁶ g/mL. HQ extract also displayed ERα activity, resulting in a maximal 8-fold increase (comparable to only 50% maximal activity of estradiol) in ERα activity at a dose of 300×10⁻⁶ g/ml. The EC₅₀ for both ERα and ERβ, were about 130×10⁻⁶ g/ml and 50×10⁻⁶ g/ml, respectively.

### Example 6

### Synergistic Effects of HQ on AR, PR and GR Activities in the Presence of Saturating Doses of the Cognate Hormones

HQ extract was prepared with ethanol as described in Example 1. Cells were exposed to increasing concentrations of HQ extract in the presence of fixed saturating concentrations of (1) dihydrotestosterone, 1×10⁻⁹ M; (2) progesterone, 1×10⁻⁶M; (3) hydrocortisone, 1×10⁻⁹ M or (4) Estradiol 1×10⁻⁹ M to test for inhibitory or synergistic effect on (1) AR, (2) PR, (3) GR or (4) ER reporter gene systems respectively, as described in the previous examples.

Although HQ extract did not have any agonist action on AR, PR and GR reporter gene assays, the extract significantly augmented the activity of saturating doses of dihydrotestosterone, progesterone and hydrocortisone on the corresponding reporter gene assays (Table 3). Thus HQ dose dependently doubled the activity of DHT and hydrocortisone on AR and GR respectively. It also increased the activity of progesterone by 140%.

HQ extract was able to increase the action of androgens, progestogens and hydrocortisone even though the hormones are at saturating doses. This property of HQ can be used to augment the endogenous or exogenous actions of androgens, progestogens and glucocorticoids to treat conditions requiring an augmentation of activity by these receptors. Examples of conditions requiring an augmentation of activity by androgens are male infertility, chronic bone and muscle mass loss, geriatric andropause, androgen insensitivity syndromes, Klinefelter syndrome and cryptorchidism; by progestogens are postmenopausal hormone replacement therapy, female infertility, endometrial cancer, secondary amenorrhea, functional uterine bleeding as well as related menstrual disorders (caused by hormonal deficiency or imbalance); by glucocorticoids are autoimmune diseases, arthritis, post-operative graft rejection and asthma.

TABLE 3
Synergistic effects of HQ extract on liganded-AR, PR and GR
reporter gene systems. All wells contain saturating doses of the
cognate hormones and the indicated doses of HQ extract

| | Luciferase activity (Fold ± SE) | | |
|---|---|---|---|
| | | Progesterone | Glucocorticoid |
| HQ extract | Androgen Receptor/ | Receptor/ | Receptor/ |
| concentration | Dihydrotestosterone | Progesterone | Hydrocortisone |
| (10⁻⁶ g/mL) | (1 × 10⁻⁹ M) | (1 × 10⁻⁶ M) | (1 × 10⁻⁹ M) |
| 0 | 328 ± 17 | 459 ± 79 | 11014 ± 285 |
| 1 | 339 ± 14 | 554 ± 78 | 17348 ± 874 |
| 3 | 409 ± 21 | 583 ± 74 | 18993 ± 676 |
| 10 | 431 ± 22 | 656 ± 54 | 20821 ± 605 |
| 30 | 655 ± 38 | 652 ± 28 | 24602 ± 1263 |

| HQ extract | Luciferase activity (Fold ± SE) | | |
|---|---|---|---|
| concen- | Androgen Receptor/ | HQ extract | Androgen Receptor/ |
| tration | Dihydrotestosterone | concentration | Dihydrotestosterone |
| (10⁻⁶ g/mL) | (1 × 10⁻⁹ M) | (10⁻⁶ g/mL) | (1 × 10⁻⁹ M) |
| 100 | 702 ± 60 | 578 ± 47 | 14210 ± 1419 |
| 300 | 521 ± 22 | 291 ± 20 | 14632 ± 294 |

### Example 7

### Methods for Preparing Extracts of Astragalus Membranaceus Enriched for PPAR Active Compound(s)

Astragalus Membranaceus extract (HQ extract) was prepared as described in Example 1, except that the temperature of maceration was changed from 20% to 100%. The PPAR activity of HQ extract was not inactivated by heat suggesting that PPAR active compound(s) in HQ could be heat resistant up to 100° C. Astragalus Membranaceus extracts (HQ extract) were prepared using a Soxhlet extractor for 4 hours with solvents of varying polarity; hexane, dichloromethane (DCM), chloroform and ethanol. The HQ extracts obtained were tested for PPAR activity as described in Example 1 (Tables 4 and 5). In such a system, compounds with strong PPARγ activity were detected at indicated concentrations mainly in the highly non-polar hexane and DCM fractions, with lesser activity observed in the chloroform fraction, and minimal activity with tithanol. Referring to Table 4 and FIG. 1, HQ ethanolic extracts at concentrations 1 to 30 μg/mL had very minimal activity but significant activity was observed at 300 μg/mL (Table 4).

The hexane, DCM and chloroform fractions are also enriched for compounds with PPARα activity (Table 5). Maximal activity was observed at a dose of 30×10⁻⁶ g/ml, where PPARγ and PPARα activities were increased by 16- and 8-fold, respectively, i.e., both hexane and DCM extracts exhibited a 16-fold on PPARγ and 18-fold on PPARα. PPARα and PPARγ bioactive fractions can also be extracted using water by Soxhlet extraction but for duration of 16 hours. The water extract dose-dependently increased PPARγ activity reaching a maximal 18-fold stimulation at a dose of 8×10⁻³ g/ml (Table 6). Thus the dose-response curve for the water extract was shifted to the right with respect to hexane, DCM and chloroform fractions, indicating that the water extract contained a lesser concentration of the active compounds.

For example, by referring to Tables 4, 5 and 6, the maximal PPAR activity for hexane and bCM extracts are observed at a concentration of 0.03 mg/mL, but the maximal activity for water extracts was only observed at 8 mg/mL. This indicates a concentration-activity shift where the Water extract is less potent compared to hexane and DCM extracts. This is also indicated by the EC50 values in Tables 4, 5 and 6.

The yield of extract in percentage weigh/weight with water was 50%. In comparison, the yields with hexane, DCM and ethanol were 0.8%, 1.2% and 16% respectively. Thus water, DCM, ethanol and hexane can be used as solvents to extract PPAR bioactive compounds, and the techniques above can be used to obtain herbal drugs with PPAR agonistic activity. Since the hexane and dichloromethane extracts were about 10 times more potent than the ethanolid extract, these solvents could be used to prepare extracts enriched for bPAR active compounds. Moreover, formononetin and calycosin concentrations in HQ extracts were analyzed using Liquid Chromatography—Mass Spectrometry-Mass spectrometry (LC-MS-MS) (Table 7) and both compounds $ere found to be highest in HQ DCM extracts. Further, it can be derived (based on data in Tables 4-7) that the hexane extract was enriched with formononetin and calycosin. For example, the highest concentration of calycosin was found in DCM extract (Table 7) and this extract was also very active (ECS0 of 15, as seen in Table 4). Since the hexane extract is also active, it was concluded that calycosin was found in high concentrations in the extract.

Thus HQ hexane, DCM and chloroform extracts are activators of PPARγ and therefore can be used in the treatment of PPARγ-mediated conditions. Based on Table 4, doses of HQ hexane or chloroform extract resulting in 1 to 30×10⁻⁶ g/mL of HQ in plasma indicate to have a therapeutic effect. Considering 100% absorption in an adult human with a normal blood volume of 5 L, doses of 5 to 150×10⁻³ g of HQ hexane or chloroform extract is required for a clinical effect. Thus HQ can be used to treat conditions mediated by PPARγ like diabetes, polycystic ovarian disease, cancer, and inflammatory bowel disease.

HQ hexane, DCM and chloroform extracts were also activators of PPARα. As can be seen in Table 5, PPARα activity was first observed at a dose 1×10⁻⁶ g/mL, rising to a peak at 30×10⁶ g/mL. Therefore, effective doses of HQ hexane or chloroform extract result in 1 to 30×10⁶ g/mL of HQ in plasma to observe a therapeutic effect. Considering 100% absorption in an adult human with a normal blood volume of 5 L, doses of 5 to 150×10⁻³ g of HQ is required for a therapeutic effect. Thus HQ can be used to treat conditions mediated by PPARα like dyslipidemia, atherosclerosis, coronary heart disease and obesity.

TABLE 4
PPARγ activity of HQ extracts obtained by extraction with
various solvents; at indicated concentrations

| | PPARγ activity (fold increase over vehicle ± SE) | | | | |
|---|---|---|---|---|---|
| Solvent used to | Concentration of extract (×10⁻⁶ g/mL) | | | | EC₅₀ |
| prepare extract | 1 | 3 | 10 | 30 | (×10⁻⁶ g/mL) |
| Hexane | 1.15 ± 0.15 | 2.68 ± 0.12 | 5.00 ± 0.32 | 16.4 ± 1.65 | 15 |
| Dichloromethane | 1.00 ± 0.04 | 2.67 ± 0.36 | 4.13 ± 0.35 | 15.9 ± 1.41 | 15 |
| Chloroform | 1.00 ± 0.08 | 0.92 ± 0.09 | 2.07 ± 0.18 | 3.43 ± 0.57 | 30 |
| Ethanol | ND | ND | 1.36 ± 0.05 | 1.75 ± 0.51 | 105 |

ND: Not Determined

TABLE 5
PPARα activity of HQ extracts obtained by extraction with
various solvents; at indicated concentrations

| | PPARα activity (fold increase over vehicle ± SE) | | | | |
|---|---|---|---|---|---|
| Solvent used to | Concentration of extract (×10⁻⁶ g/mL) | | | | EC₅₀ |
| prepare extract | 1 | 3 | 10 | 30 | (×10⁻⁶ g/mL) |
| Hexane | 1.60 ± 0.21 | 1.64 ± 0.18 | 5.62 ± 0.72 | 6.76 ± 0.27 | 6 |
| Dichloromethane | 1.00 ± 0.05 | 1.72 V 0.06 | 3.67 ± 0.15 | 8.98 ± 2.12 | 11 |
| Chloroform | 1.00 ± 0.20 | 1.19 ± 0.27 | 2.04 ± 0.25 | 3.06 ± 0.33 | 100 |
| Ethanol | ND | ND | 2.18 ± 0.10 | 2.47 ± 0.28 | 100 |

ND: Not Determined

TABLE 6
PPARγ and PPARα activity of HQ extracts obtained
by extraction with Water; at indicated concentrations

| | |
|---|---|
| | PPAR activity |
| | (fold increase |
| Concentration of | over vehicle ± SE) |

| extract (×10⁻³ g/mL) | PPARγ | PPARα |
|---|---|---|
| 0.0 | 1.00 ± 0.07 | 1.00 ± 0.19 |
| 0.5 | 3.37 ± 0.49 | 2.54 ± 0.21 |
| 1.0 | 4.31 ± 0.26 | 6.97 ± 0.14 |
| 2.0 | 6.78 ± 0.42 | 10.5 ± 0.33 |
| 4.0 | 8.94 ± 1.26 | 15.4 ± 2.43 |
| 8.0 | 18.5 ± 2.39 | 29.9 ± 3.81 |
| EC₅₀ (×10⁻⁶ g/mL) | 4000 | 4000 |

TABLE 7
Formononetin and Calycosin Concentrations by LC-MS-MS analysis

| | |
|---|---|
| Solvent used to | Concentration (×10⁻³ g/g) |

| prepare extract | Formononetin | Calycosin |
|---|---|---|
| Dichloromethane | 1.55 | 0.55 |
| Ethanol | 1.38 | 0.31 |
| Water | 0.63 | 0.05 |

### Example 8

### Methods for Fractionation and Isolation of PPAR Active Compound(s)

Bioassay-guilded fractionation was performed to identify the bioactive compound(s). PPAR activity was assayed as described in Example 1. Dried roots of Astragalus Membranaceus (—8 Kg) were milled into small pieces and then extracted with DCM. The extract was dried at 40° C. under vacuum to give an oily brown-color extract (—90 g). 10 g of the DCM extract was dry packed with 15 g of silica gel (LiChroprep, Si 60 40˜63 μm) and applied onto the top of a Medium Pressure Liquid Chromatography glass column packed under pressure with 350 g silica gel (LiChroprep, Si 60 15˜25 μm). The column was successively eluted using mixtures of Hexane and Acetone with increasing polarity as follows: 99:1, 98:2, 97:3, . . . , 90:10, 80:20, . . . , 10:90, 0:100, and in the end washed with methanol. Among the 37 fractions collected by the MPLC system, Compound I and Compound II were obtained from fraction 19 (70:30; Hexane:Acetone) (72.2 mg) and 29 (60:40; Hexane:Acetone) (26.3 mg), respectively. Thus this is an effective method for obtaining fractions that are enriched for Compound I and Compound II.

### Example 9

### Structure Elucidations of Compound I

Compound I was obtained as fine white needles with a melting point of 257-258° C. (Hexane/Acetone). Purity analysis using reverse-phase HPLC indicated that it is≧99% pure. Its HREIMS showed the [M]+at m/z 268.0729, consistent with a molecular formula of C₁₆H₁₂O₄. Analysis of the ¹H and ¹³C NMR spectra showed the characteristic chemical shifts for a 7,4′-disubstituted isoflavone. In the ¹H NMR spectrum, H-2 appeared as a single peak at the lowest field; H-6 (δ6.92) coupling with both H-5 (δ795, d) and H-8 (δ6.85, d), appeared as a typical dd peak (J=8.8, 2.35 Hz). The highly structural symmetry of B ring caused the signals of H-3′ and 5′ to resonate as one group of multiple peaks (δ6.96) in the low field, followed by those of H-2′ and H-6′ (δ7.50, m). In the high field, the methyl of 4′OCH₃ showed a sharp single peak at δ 3.79. The ¹³C-NMR spectral data (in ppm) are as follows: C2-153.0, C3-124.3, C4-174.6, C5-127.2, C6-115.4, C7-163.0, C8-102.1, C9-157.5, C10-116.4, C1′-123.1, C2′-130.0, C3′-113.6, C4′-158.9, C5′-113.6, C6′-130.0, OCH₃-55, which are identical to those of the reference (Carbon-13 NMR of Flavonoids, by P. K. Agrawal). According to the above analysis, the structure of Compound I can be determined as 7-Hydroxy-4′-methoxyisoflavone, which is a known phytoestrogen, named as formononetin.

The structure of formononetin is:

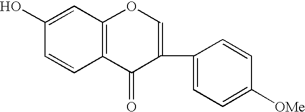

### Example 10

### Structure Elucidations of Compound II

Compound II was obtained as long white needles with a lower melting point at 251-252° C. (Hexane/Acetone). Analysis of its HREIMS ([M]+m/z 284.0685) indicated the molecular formula was C₁₆H₁₂O₅, which had one more “O” atom than that of formononetin. The ¹H, ¹³C NMR spectra also showed the characteristic, chemical shifts of isoflavone, but the signals of B ring were quite different from those of formononetin. In the ¹H NMR spectrum, because of the substitution of one more —OH group to B ring, the structural symmetry was corrupted, and accordingly some proton signals resonated to different positions. In detail, H-2′ (δ7.0. d, J=1.85 Hz) appeared as a broad d peak in the low field followed by the highly overlapped signals of H-5′ and H-6′ at δ6.94. Compared with the reference, the ¹³C-NMR spectral data (in ppm) are determined as follows: C2-152.9, C3-124.7, C4-174.5, C5-127.1, C6-115.3, C7-163.1, C8-102.0, C9-157.4, C10-116.4, C1′-123.3, C2′-116.3, C3′-146.0, C4′-147.4, C5′-112.0, C6′-119.6, OCH₃-55.6. Finally, the structure of Compound I was characterized as 3′,7-Dihydroxy-4′-methoxyisoflavone, which is also a known compound, named as calycosin.

The structure of calycosin is:

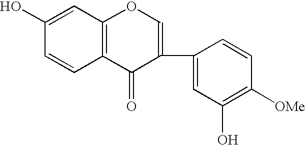

### Example 11

### Agonistic Action of Formononetin on PPARγ and PPARα, and ERα and ERβ

The bioassay was performed as that described in the previous examples. Formononetin was diluted in dimethylsulfoxide (DMSO) and tested at concentrations 3, 10 and 30×10⁻⁶ g/mL. Formononetin displayed strong agonist activities with ERα, ERβ, PPARγand PPARα. The presence of formononetin resulted in a 17-fold increase (comparable to maximal activity of Estradiol) in ERα activity at a concentration of 30×10⁻⁶ g/ml. Formononetin also displayed strong ERP activity, resulting in a maximal 130-fold increase (comparable to 400% maximal activity of Estradiol) at a dose of 30×10⁻⁶ g/ml. The activity of formononetin on the other receptors are as described in Table 8, expressed as fold increase in luciferase activity compared to cells exposed to vehicle only. Thus formononetin increased PPARγ and PPARα activities by up to 6- and 10-fold respectively (Table 8). Thus formononetin would be useful to increase PPARγ and PPARα activities in diseases associated with deficiencies in the activity of these receptors.

TABLE 8
Formononetin as an agonist for PPARγ and PPARα and ER

| Concen- | Luciferase activity (Fold increase | | | |
|---|---|---|---|---|
| tration | over vehicle ± SE) | | | |
| (10⁻⁶ g/mL) | PPARγ | PPARα | ERα | ERβ |
| 0 | 1.00 ± 0.07 | 1.00 ± 0.09 | 1.00 ± 0.03 | 1.00 ± 0.102 |
| 1 | 2.11 ± 0.31 | 3.12 ± 0.01 | ND | ND |
| 3 | 1.30 ± 0.18 | 2.97 ± 0.55 | 5.99 ± 0.53 | 62.7 ± 8.66 |
| 10 | 5.48 ± 1.30 | 4.24 ± 0.01 | 11.5 ± 3.17 | 89.4 ± 29.5 |
| 30 | 6.96 ± 0.07 | 10.4 ± 0.7 | 16.8 ± 1.20 | 131 ± 24.0 |

ND: Not Determined

### Example 12

### Synergistic Action of Formononetin on PPARγ and PPARα, AR, PR and ERαR and ERβ

HeLa cells were exposed to increasing concentrations of formononetin diluted in DMSO in the presence of fixed concentrations of (1) pioglitazone, (30×10⁻⁶M); (2) WY14643 (30×10⁻⁶M); (3) dihydrotestosterone, 1×10⁻⁹M; (4) progesterone, 10×10⁻⁶M; or (5) estradil (1×10⁻⁹) to test for inhibitory or synergistic effect on (1) PPARγ, (2) PPARα, (3) AR. (4) PR, or (5) ER reporter gene system respectively. These concentrations are at the plateau of dose-response curves observed with the respective receptors. Full details of these reporter gene assays have been described in as described in the previous examples.

Unexpectedly, formononetin augmented the activity of WY14643, dihydrotestosterone, progesterone and Estradiol on the PPARα, AR, PR and ERα reporter gene assays respectively (Table 9). Thus formononetin was able to synergize and increase the activity of maximal doses of PPAR agonist by 4.4-fold, AR agonist by 2-fold, PR agonist by 1.8-fold, and ERα by 3.2-fold. In contrast, the PPARγ activity of formononetin (at doses ranging from 3 to 30×⁻⁶ g/mL) when added to pioglitazone (30×10⁻⁶M) was no different than that observed with pioglitazone alone. In addition, the ERβ activity of formononetin (at doses ranging from 1 to 30×10⁻⁶ g/mL) when added to estradiol (1×10⁻⁹M) exhibited its own potent estrogenic activity. Thus formononetin, at doses ranging from 1 to 30×10⁻⁶ g/mL, did not inhibit or augment the activity of estradiol alone. Those skilled in the art will realize that this novel augmentative ability of formononetin can be utilized to boost the effects of endogenous or exogenous androgens, estrogens, progestogens and PPARα ligands in diseases due to deficiency in these hormones. It can also be used to augment the effects of these hormones in normal people where such boosting effects are desired.

TABLE 9
Synergistic effects of formononetin on PPAR, AR, PR and ER reporter gene systems

| Luciferase activity | Formonenetin concentration (10⁻⁶ g/mL) | | | |
|---|---|---|---|---|
| (Fold over vehicle ± SE) | 0 | 0.3 | 1 | 3 |
| PPARα/WY14643 (30 × 10⁻⁶M) | 14.9 ± 1.92 | 35.6 ± 2.63 | 43.7 ± 2.60 | 66.0 ± 3.21 |
| Luciferase activity | Formonenetin concentration (10⁻⁶ g/mL) | | | |
| (Fold over vehicle ± SE) | 0 | 3 | 10 | 30 |
| PR/Progesterone (10 × 10⁻⁶M) | 364 ± 65.3 | 654 ± 120 | 564 ± 65.6 | 618 ± 44.7 |
| ERα/Estradiol (1 × 10⁻⁹M) | 17.6 ± 3.74 | 55.5 ± 4.62 | 52.8 ± 4.72 | 47.3 ± 10.7 |
| Erβ/Estradiol (1 × 10⁻⁹M) | 33.0 ± 3.52 | 86.2 ± 7.35 | 113 ± 14.2 | 110 ± 13.7 |

### Example 13

### Agnostic Action of Calycosin on PPAγ, ERα and ERβ

Bioassays were performed as that described in the previous examples. Calycosin was diluted in dimethylsulfoxide (DMSO) and tested at concentrations of 3, 70 and 30×10⁻⁶ g/mL. Calycosin displayed strong PPARγ activity resulting in a maximal 40-fold (comparable to 50% maximal activity of pioglitazone on PPARγ) at a concentration of 30×10⁻⁶ g/ml. In addition, calycosin also exhibit ERα agonistic activity, resulting in a maximal 20-fold increase (comparable to 50% maximal activity of Estradiol on ERα) at a concentration of 30×10⁻⁶ g/ml. Similarly, at a concentration of 30×10⁻⁶ g/ml, calycosin displayed strong ERβ activity, resulting in a maximal 100-fold increase (comparable to 400% maximal activity of Estradiol on ERβ). Thus calycosin can be used to increase PPARγ, ERα and ERβ activities in diseases associated with deficiencies in the activity of these receptors. It is also a SERM since it activates ERβ more than ERα.

### Example 14

### Synergistic action of Calycosin on PPARγ and PPARγ, AR, PR and ERα and ERβ

Cells were exposed to increasing concentrations of calycosin diluted in DMSO in the presence of fixed concentrations of (1) pioglitazone, (30×10⁻⁶M); (2) WY14643 (30×10⁻⁶); (3) dihydrotestosterone, 1×10⁻⁹M; (4) progesterone, 10××10⁻⁶M; or (5) estradiol (1××10⁻⁹M) to test for inhibitory or synergistic effect on (1) PPARα, (3) AR, (4) PR, or (5) ER reporter gene systems respectively, as described in the previous examples.

Unexpectedly, calycosin augmented the activity of pioglitazone, WY14643, dihydrotestosterone, progesterone and estradiol on the PPARγ, PPARα, AR, PR and ERα reporter gene assays respectively (Table 10). These concentrations are at the plateau of dose-response curves observed with the respective receptors. Thus calycosin was able to synergize and increase the activity of maximal doses of PPARγ agonist by 3-fold, PPARα agonist by 1.8-fold, AR agonist by 5.8-fold, PR agonist by 1.8-fold, and ERα by 2.9-fold. The effect of calycpsin on ERP activity was additive at concentrations ranging from 1 to 30×10⁻⁶ g/mL, increasing the effect of estradiol (1×10⁻⁹M) due to its own potent estrogenic activity. Those skilled in the art will realize that this novel augmentative ability of calycosin can be utilized to boost the effects of endogenous or exogenous PPARα and PPARγ, ligands, androgens, estrogens, and progestogens in diseases due to deficiency in these hormones. It can also be used to augment the effects of these hormones in normal people where such boosting effects are desired.

TABLE 10
Synergistic effects of calycosin on PPAR, AR, PR and ER reporter gene systems

| Luciferase activity | Calycosin concentration (10⁻⁶ g/mL) | | | |
|---|---|---|---|---|
| (Fold over vehicle ± SE) | 0 | 3 | 10 | 30 |
| PPARγ/Pioglitazone (30 × 10⁻⁶M) | 30.4 ± 24.0 | 105 ± 10.7 | 162 ± 28.5 | 253 ± 30.7 |
| PPARα/WY14643 (30 × 10⁻⁶M) | 162 ± 31.7 | 197 ± 20.6 | 257 ± 8.85 | 294 ± 39.2 |
| AR/ | 771 ± 27.6 | 1997 ± 96.1 | 4080 ± 5.67 | 4502 ± 995 |
| Dihydrotestosterone (1 × 10⁻⁹M) | | | | |
| PR/Progesterone (10 × 10⁻⁶M) | 344 ± 13.8 | 1077 ± 43.4 | 943 ± 44.0 | 837 ± 115 |
| ERα/Estradiol (1 × 10⁻⁹M) | 39.4 ± 0.33 | 118 ± 2.18 | 127 ± 6.3 | 114 ± 4.18 |
| ERβ/Estradiol (1 × 10⁻⁹M) | 26.7 ± 2.60 | 55.2 ± 1.21 | 65.1 ± 2.71 | 70.7 ± 3.61 |

### Example 15

### Agonistic Action of Daidzein on PPARγ, PPARα, ERα and ERβ

Bioassays were performed as that described in previous examples. Daidzein was diluted in dimethylsulfoxide (DMSO) and tested at concentrations of 5, 10 and 20×10⁻⁶ g/mL. Daidzein displayed PPARγ activity resulting in a maximal 20-fold (comparable to 22% maximal activity of pioglitazone on PPARγ) at a concentration of 20×10⁻⁶ g/m. Daidzein also has PPARα activity with a maximal 8-fold (comparable to 73% maximal activity of W14643 on PPARγ) at a concentration of 10×10⁻⁶ g/ml. In addition, daidzein exhibits ERα agonistic activity, resulting in a maximal 20-fold increase (comparable to 100% maximal activity of Estradiol on ERα) at a concentration of 5×10⁻⁶ g/ml. Similarly, at a concentration of 30×10⁻⁶ g/ml, daidzein displayed strong ERβ activity, resulting in a maximal 29-fold increase (comparable to 200% maximal activity of Estradiol on ERβ). Thus daidzein can be used to increase PPARα and γ, ERα and ERβ activities in diseases associated with deficiencies in the activity of these receptors. It is also a SERM since it activates ERβ more than ERα.

### Example 16

### Synergistic Action of Daidzein on PPARα, AR and PR

HeLa cells were exposed to increasing concentrations of daidzein diluted in DMSO in the presence of fixed concentrations of (1) pioglitazone, (30×10⁻⁶ M); (2) WY14643 (30×10⁻⁶M); (3) dihydrotestosterone, 1×10⁻⁹M; (4) progesterone, 100×10⁻⁹M; or (5) estradiol (1×10⁻⁹M) to test for inhibitory or synergistic effect on (1) PPARγ, (2) PPARα, (3) AR, (4) PR, or (5) ER reporter gene systems respectively, as described in Examples 1 and 4.

Unexpectedly, daidzein augmented the activity of WY 14643, dihydrotestosterone, progesterone on the PPARα, AR and PR reporter gene assays respectively (Table 11). These concentrations are at the plateau of dose-response curves observed with the respective receptors. Thus calycosin was able to synergize and increase the activity of maximal doses of PPARα agonist by 7 fold, AR agonist by 3-fold and PR agonist by 3-fold. Those skilled in the art will realize that this novel augmentative ability of daidzein can be utilized to boost the effects of endogenous or exogenous PPARα ligands, androgens, and progestogens in diseases due to deficiency in these hormones. It can also be used to augment the effects of these hormones in normal people where such boosting effects are desired.

TABLE 11
Synergistic effects of daidzein on PPAR, AR, PR reporter gene systems

| Luciferase | | | | |
|---|---|---|---|---|
| activity | | | | |
| (Fold over | Daidzein concentration (10⁻⁶ g/mL) | | | |
| vehicle ± SE) | 0 | 5 | 10 | 20 |
| PPARα/ | 10.9 ± 1.33 | 58.2 ± 14.9 | 71.3 ± 9.90 | 53.4 ± 13.3 |
| WY14643 | | | | |
| (30 × 10⁻⁶M) | | | | |
| AR/ | 657 ± 15.1 | 1703 ± 154 | 1684 ± 146 | 1547 ± 257 |
| Dihydro- | | | | |
| testosterone | | | | |
| (1 × 10⁻⁹M) | | | | |
| PR/ | 220 ± 54.2 | 569 ± 84.0 | 607 ± 90.6 | 326 ± 65.8 |
| Progesterone | | | | |
| (10 × 10⁻⁹M) | | | | |

### Example 17

### Synergistic action of Genistein on AR and Other Steroid Receptors

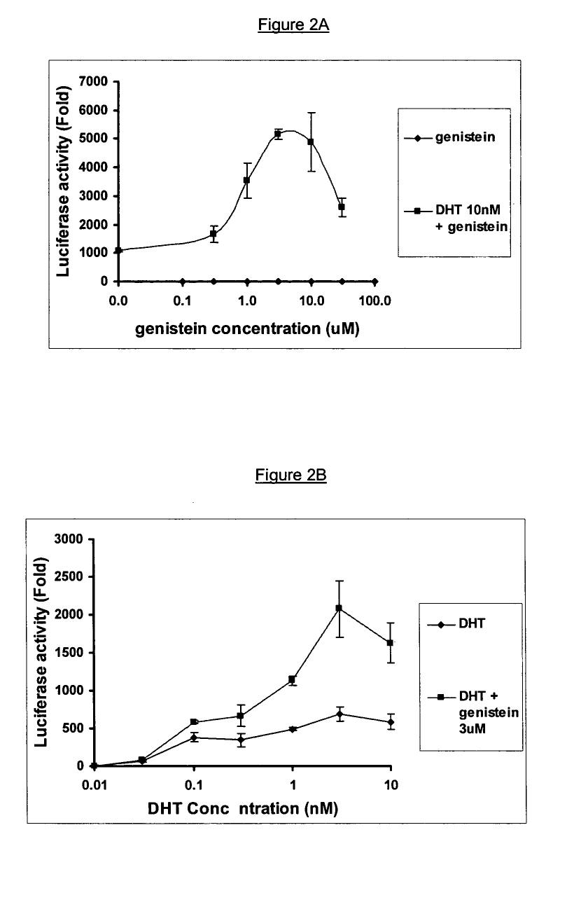

To measure the effects of genistein on liganded-AR activity, HeLa cells were exposed to increasing concentrations of genistein diluted in ethanol in the absence or presence of fixed concentrations of dihydrotestosterone, (10×10⁻⁹M) (FIG. 2A). Concentrations of dihydrotestosterone above 3×10⁻⁹M results in maximal androgenic action and a plateau in the dose-response curve. Cells were also exposed to increasing concentrations of dihydrotestosterone in the absence, or presence, of fixed concentrations of genistein (3×10⁻⁶M) (FIG. 28). Androgenic activity was measured with an AR-driven reporter gene assay as described in Example 5. Although genistein (at concentrations ranging from 0.1 to 30×10⁻⁶M) does not display any intrinsic agohistic AR activity, it was able to further increase by 5-fold the activity of sbturating doses of dihydrotestosterone in a dose-responsive manner (FIG. 2A). A fixed concentration of Genistein at 3×10⁻⁶ M also augmented the activity of dihydrotestosterone on AR at concentrations ranging from 0.01 to 10×10⁻⁹M (FIG. 2B). This synergistic action of genistein was not due to an increase in AR protein expression as immunoblot analyses using replicate HeLa cell lysates, in the presence of DHT, with or without genistein, did not show any differences in expression of AR protein. Genistein, due to the similar isoflavonoid backbone as formononetin, daidzein and calycosin, may also boost the action of PPARα and PPARγligands, estrogens, and progestogens. This novel augmentative ability of genistein, a commonly consumed soy isoflavone, can be used to boost the effects of endogenous or exogenous PPARα and PPARγ ligands, androgens, estrogens, and progestogens in diseases due to deficiency in these hormones. It can also be used to augment the effects of these hormones in normal people where such boosting effects are desired.

### Example 18

### Effect of the PPARγ Antagonist, GW9662, on the Synergistic Action of Extracts and Compounds from HQ

Since the synergistic effects of extracts and compounds from HQ were not due to increased expression of steroid receptors, we performed experiments to determine if these effects were mediated through their tigand binding pocket. GW9662 is a mixed agonist/antagonist which binds irreversibly to the ligand-binding pocket of PPAR (Leesnitzer et al., 2002). On its own, GW9662 displayed weak PPARγ agonistic activity (Table 12). As expected, the presence of GW9662, at doses above 1×10⁻⁶M inhibited the PPARγ effects of pioglitazone in a dose-dependent manner. Unexpectedly, the presence of HQ water extract, HQ DCM extract, formononetin, genistein were able to increase the intrinsic agonist and antagonistic activity of GW9662. Thus an augmented PPARγ stimulatory effect was observed when extracts and compounds of HQ were added to low doses (0.3 to 1.0×10⁻⁶M) of GW9662 (Table 13). Higher doses of GW9662 (1×10⁶M) resulted in an antagonistic action similar to that observed with pioglitazone. These agonist/antagonist actions of GW9662 were thus shifted upwards indicating that the augmentative effects of extracts and compounds of HQ and genistein were not mediated through the ligand-binding pocket of PPARγ. Because GW9662 binds irreversibly to the ligand-binding pocket of PPARγ, extracts and compounds of HQ and genistein exert their augmentative effect through mechanism(s) that do NOT involve the ligand pocket of PPARγ. More generally, we have discovered new method(s) to augment the action of steroid receptors such as AR, PR GR and ER that does not involve specific ligand binding to the ligand-binding pocket of steroid receptors. This discovery allows the development of new drug discovery screening platforms to search for compounds that may augment the action of liganded-steroid receptors.

TABLE 12
Effect of increasing doses of GW9662 alone and in combination
with a fixed dose of pioglitazone. PPARγ activity was
measured as described in Example 1

| | Luciferase activity (% Pioglitazone | |
|---|---|---|
| | 30 × 100⁻⁶M ± SE | |
| Concentration of GW9662 | Vehicle (i.e., | Piogliltazone |
| (×10⁻⁶M) | dimethylsulfoxide, DMSO) | (30 × 10⁻⁶M) |
| 0 | 0.543 ± 0.190 | 100 ± 21.2 |
| 0.1 | 13.8 ± 0.207 | 97 ± 23.8 |
| 0.1 | 13.8 ± 2.28 | 114 ± 5 |
| 1 | 23.5 ± 2.74 | 77.2 ± 11.4 |
| 10 | 39.3 ± 4.38 | 40.6 ± 12.3 |

TABLE 13
Effect of increasing doses of GW9662 on PPARα activities
of fixed doses of HQ extracts, formonenetin and genistein. PPAR
activity was measured as described in Example 1
Luciferase activity (Fold ± SE)

| Concentration | HQ Water | | | |
|---|---|---|---|---|
| of GW9662 | extract | HQ DCM extract | Formononetin | Genistein |
| (×10⁻⁶M) | (8 × 10⁻⁶ g/mL) | (50 × 10⁻⁶ g/mL) | (10 × 10⁻⁶ M) | (10 × 10⁻⁶ M) |
| 0 | 28.8 ± 3.92 | 15.1 ± 0.76 | 4.78 ± 0.184 | 7.37 ± 0.128 |
| 0.3 | 43.7 ± 9.97 | 61.2 ± 6.86 | 91.5 ± 1.54 | 93.6 ± 2.47 |
| 1 | 73.1 ± 13.2 | 56.1 ± 2.54 | 106 ± 8.28 | 83.9 ± 4.37 |
| 3 | 44.2 ± 7.93 | 62.2 ± 1.73 | 94.4 ± 3.24 | 82.5 ± 2.63 |
| 10 | 61.5 ± 28.3 | 55.0 ± 4.01 | 69.1 ± 0.575 | 67.4 ± 1.52 |
| 30 | 20.4 ± 3.54 | 30.8 ± 1.44 | 44.8 ± 1.10 | 39.5 ± 0.403 |

### Bibliography

Agarwal, P. K. (1989) “Carbon-13 NMR Of Flavanoids,” Elsevier Health Sciences.

Brinkmann, A. O., Faber, P. W., van Rooij, H. C., Kuiper, G. G., R is, C., Klaasen, P., van der Korput, J. A., Voorhorst, M. M., van Laar, J. H., Mulder, E., et al. (1989) “The Human Androgen Receptor: Domain Structure, Genomic Organization And Regulation Of Expression,” J Steroid Biochem. 34(1-6):307-10.

ElSohly, H. N., Joshi, A. S., Nimrod, A. C. (1999) “Antigiardial Isoflavones From Machaerium Aristulatum,” Planta medica 65:490.

Gould Rothbbrg, B. E., Sundseth, S. S., DiPippo, V. A., Brown, P. J., Winegar, D. A., Gottshalk, W. K., Shenoy, S. G., Rothberg, J. M. (2001) “The Characterization Of PPAR Alpha Ligand Drug Action In An In Vivo Model By Comprehensive Differential Gene Expression Profiling,” Funct. Integr. Genomics 1(5):294-304.

Haubenwallner, S., Essenburg, A. D., Barnett, B. C., Pape, M. E., DeMattos, R. B., Krause, B. R., Minton, L. L., Auerbach, B. J., Newton, R. S., Leff, T., et al. (1995) “Hypolipidemic Activity Of Select Fibrates Correlates To Changes In Hepatic Apolipoprotein C-III Expression: A Potential Physiologic Basis For Their Mode Of Action,” J. Lipid Res. 36(12):2541-51.

He, Z. Q. and Wang, B. Q. (1990) Acta Pharmaceutica Sinica, 25(9):694-8.

Jain, A. C., Pyare, L. A. L., Seshadri, T. R. (1969) “Synthesis Of 3′-Hydroxyformononetin,” Indian Journal of Chemistry 7(3): 305-6.

Keiji, K., Yasuhiro, T., Keiichi, M., Yoshiyuki, S., Mayumi, M., Yoshikazu, S., Hiroshi, Y., Kaisuke, Y. (1997) “Polysaccharide Of Astragali Radix Enhances Igm Antibody Production In Aged Mice”, “Biol. Pharm. Bull. 20(11):1178-82.

Kersten, S., Desvergne, B., Wahli, W. (2000) “Roles Of Ppars In Health And Disease,” Nature 405:421.

Koeffler, H. P. (2003) “Peroxisome Proliferator-Activated Receptor Gamma And Cancers,” Clin. Cancer Res. 9(1):1-9.

Kraft, C., Jenett-Siems, K., Siems, K., Gupta, M. P., Bienzle, U., Eich, E. (2000) “Antiplasmodial Activity Of Isoflavones From Andira Inermis,” Journal of Ethnopharmacology 73:131-5.

Kuiper, G. G. J. M., Josephine, G. L., Carlsson, B., Corton, J. C., Safe, S. H., Saag, P. T., Burg, B., Gustafsson, J. (1998) “Interaction Of Estrogenic Chemicals And Phytoestrogens With Estrogen Receptor β,” Endocrinology 139(10):4252-63.

Leesnitzer, L. M., Parks, D. J., Bledsoe, R. K., Cobb, J. E., Collins, J. L., Consler, T. G., Davis, R. G., Hull-Ryde, E. A., Lenhard, J. M., Patel, L., Plunket, K. D., Shenk, J. L., Stimmel, J. B., Therapontos, C., Wilson, T. M., Blanchard, S. G. (2002) “Functional Consequences Of Cysteine Modification In The Ligand Binding Sites Of Peroxisome Proliferator Activated Receptors By GW9662,” Biochemistry 41(21):6640-50.

Li, W., Koike, K., Asada, Y., Hirotani, M., Rui, H., Yoshikawa, T., Hikaido, T. (2002) “Flavonoids From Glycyrrhiza Pallidiflora Hairy Root Cultures,” Phytochemistry 60:351-5.

Lin, L. Z., He, X. G., Lindemnaier, M., Nolan, G., Yang, J., Cleary, M., Qiu, S. X., Cordell, G. A. (2000) “Liquid Chromatography-Electrospray Ionization Mass Spectrometry Study Of The Flavonoids Of The Roots Of Astragalus Mongholicus And A. Membranaceus,” Journal of Chromatography A 876:87-95.

Loy, C. J., Sim, K. S., Yong, E. L. (2003) “Filamin-A Localizes To The Nucleus To Regulate Androgen Receptor And Coactivator Functions,” Proceedings of the National Academy of Sciences (USA) 100:4562-4567.

Lu, J., Zou, D., Zhang, J. (1999) “Preventive Effect Of Radix Astragali On Insulin Resistance Caused By Tumor Necrosis Factor-Alpha,” Zhongguo Zhong Xi Yi Jie He Za Zhl 19(7):420-2.

Ma, J., Peng, A., Lin, S. (1998) “Mechanisms Of The Therapeutic Effect Of Astragalus Membranaceus On Sodium And Water Retention In Experimental Heart Failure,” Chin Med. J. (Engl) 111(1): 17-23.

Ma, X. Q., Shi, Q., Duan, J. A., Dong, T. T. X, Tsim, K. W. K. (2002) “Chemical Analysis Of Radix Astragali (Huangqi) In China: A Comparison With Its Adulterants And Seasonal Variations,” Journal of Agricultural and Food Chemistry 50(17):4861-6.

Ma, X., et al. (2003) J. Chromatogr., Apr. 11, 992(1-2):193-7.

Mangelsdorf, D. J., Evans, R. M. (1995) “The RXR Heterodimers And Orphan Receptors,” Cell 83:841.

Miksicek, R. J. (1994) “Interaction Of Naturally Occurring Nonsteroidal Estrogens With Expressed Recombinant Human Estrogen Receptor,” J. Steroid Biochem. Mol. Biol. 49(2-3): 153-60.

Morito, K., Aomori, T., Hirose, T., Kinjo, J., Hasegawa, J., Ogawa, S., Inoue, S., Muramatsu, M., Masamune, Y. (2002) “Interaction Of Phytoestrogens With Estrogen Receptors α and β,” Biol. Pharm. Bull. 25(1):48-52.

Muller, M., Baniahmad, C., Kaltschmidt, C., Renkawitz, R. (1991) “Multiple Domains Of The Glucocorticoid Receptor Involved In Synergism With The CACCC Box Factor(s),” Mol. Endocrinol. 5: 1498-1503.

Olsson, O., Kopez, C., Szalay, A. A. (1988) The Use Of The Iuxa Gene Of The Bacterial Luciferase Operon As A Reporter Gene,” Mol. Gen. Genet. 215(1):1-9.

“Pharmacopoeia Of The People's Republic Of China,” (1997) Chemical Industry Press, Beijing China 1:42.

“Phytochemical Dictionary Of The Leguminosae,” (1994) Vol. 1, 1st ed., Chapman & Hall, London, New York.

Pool-Zobel, B. L., Adlercreutz, H., Glei, Michael, Liegibel, U. M., Sittlingon, J., Rowland, I., Wahala, K., Rechkemmer, G. (2000) “Isoflavonoids And Lignans Have Different Potentials To Modulate Oxidative Genetic Damage In Human Colon Cells,” Carcinogenesis 21(6): 1247-52.

Qian, Z., Mao, S., Cai, X., Zhang, X., Gao, F., Lu, M., Shao, X., Li, X., Yang, X., Zhuo, Y., Shi, L., Duan, S., Hou, Y. (1990) “Viral Etiology Of Chronic Cervicitis And Its Therapeutic Response To A Recombinant Interferon,” Chinese Medical Journal 103(8):647-51.

Saltiel, A. R., Olefsky, J. M. (1996) “Thiazolidinediones In The Treatment Of Insulin Resistance And Type II Diabetes,” Diabetes 45:1661.

Shizuo, T., Yoshiaki, S. (1998) “Inhibitory Effects Of Isoflavones In Roots Of Astragalus Membranaceus Bunge (Astragali Radix) On Lipid Peroxidation By Reactive Oxygen Species,” Phytotherapy Research 12(1):59-61.

Shizuo, Yoshiaki, S. (1999) “Inhibitory Effects Of Isoflavones On Lipid Peroxidation By Reactive Oxygen Species,” Phytotherapy Research 13(2): 163-5.

Shizuo, Y., Yoshiro, Y., Yoshiaki, S. (2000) “Inhibitory Effects Of Astragali Radix, Crude Drug In Oriental Medicines On Lipid Peroxidation And Protein Oxidative Modification Of Mouse Brain Homogenate By Copper,” Phytotherapy Research 14:294-6.

Sun, A., Chiang, C. P. (2001) “Levamisole And/Or Chinese Medicinal Herbs Can Modulate The Serum Level Of Squamous Cell Carcinoma Associated Antigen In Patients With Erosive Oral Lichen Planus,” J. Oral Pathol. Med. 30:542-8.

Tcherepanova, I., Puigserver, P., Norris, J. D., Spiegelman, B. M., McDonnell, D. P. (2000) “Modulation Of Estrogen Receptor-α Transcriptional Activity By The Coactivator PGC-1,” J. Biol. Chem. 275(21): 16302-8.

Tolleson, W. H., Doerge, D. R., Churchwell, M. I., Marques, M. M., Roberts, D. W. (2002) “Metabolism Of Biochanin A And Formononetin By Human Liver Microsomes In Vitro,” Journal of Agricultural and Food Chemistry 50:4783-0.

Vegeto, E., Allan, G. F., Schrader, W. T., Tsai, M. J., McDonnell, D. P., O'Malley, B. W. (1992) “The Mechanism Of RU486 Antagonism Is Dependent On The Conformation Of The Carboxy-Terminal Tail Of The Human Progesterone Receptor,” Cell 69(4):703-13.

Wada, K. Nakajima, A., Blumberg, R. S. (2001) “Ppargamma And Inflammatory Bowel Disease: A New Therapeutic Target For Ulcerative Colitis And Crohn's Disease,” Trends Mol. Med. 7(8):329-31.

Willard, S. T., Frawley, L. S. (1998) “Phytoestrogens Have Agonistic And Combinatorial Effects On Estrogen-Responsive Gene Expression In MCF-7 Human Breast Cancer Cells,” Endocrine 8(2): 117-21.

Yang, Y., Jin, P., Guo, Q., Wu, W., Pu, S., Chen, H., Yang, J., Wang, K., Shi, J., Gong, Z., Shen, J., Peng, B. (1990) “Treatment Of Experimental Coxsackie B-3 Viral Myocarditis With Astragalus Membranaceus In Mice,” Chin. Med. J. (Engl) 103(1):14-8.

Yoshiaki, S., Masaki, T., Sanae, Y., Shizuo, T. (1997) “Antioxidative Components Isolated From The Roots Of Astragalus Membranaceus Bunge (Astragali Radix),” Phytotherapy Research 11:603-605.

Zhang, W., Wojta, J., Binder, B. (1997) “Regulation Of The Fibronolytic Potential Of Cultured Human Umbilical Vein Endothelial Cells: Astragaloside IV Down-regulates Plasminogen Activator Inhibitor-1 And Upregulates Tissue-Type Plasminogen Activator Expression,” J. Vasc. Res. 34(4):273-80.

Zhong Yao Xian Dai Yan Jiu Yu Lin Chuan Yin Yong (1993) pp. 592.

1. An extract of Astragalus Membranaceus (HQ), wherein the extract is selected from the group consisting of: a hexane extract of Astragalus Membranaceus (HQ); a dichloromethane extract of Astragalus Membranaceus (HQ); a chloroform extract of Astragalus Membranaceus (HQ); and a non-aqueous extract of Astragalus Membranaceus (HQ) provided that the HQ extract is not an ethanol or butanol HQ extract.

2. The extract of claim 1, wherein the extract is enriched in steroid/nuclear (s/n) receptors bioactives.

3. The extract of claim 1, wherein the extract is present in a composition.

4. The extract of claim 3, wherein the composition further comprises at least one flavonoid compound.

5. The extract of claim 3, wherein the composition is a pharmaceutical or dietary supplement composition for the treatment of steroid/nuclear (s/n) receptor-mediated physiological conditions.

6. The extract of claim 5, wherein the s/n receptor-mediated physiological condition is PPAR-mediated disease and is diabetes, cancer, polycystic ovarian disease, inflammatory bowel disease, dyslipidaemia, atherosclerosis, coronary heart disease or obesity.

7. The extract of claim 5, wherein the composition further comprises at least one pharmaceutically acceptable carrier and/or diluent.

8. The extract of claim 5, wherein the composition comprises an effective amount of 1 to 1000 μg/mL of extract for the treatment of PPARγ-mediated physiological conditions.

9. The extract of claim 5, wherein the composition comprises an effective amount of 1 to 1000 μg/mL of extract for the treatment of PPARα-mediated physiological conditions.

10. The extract of claim 3, wherein the composition is present in a food product or beverage.

11. The extract of claim 3, wherein the composition is present in a commercial package, and wherein the package includes instructions for use.

12. A method for the treatment of s/n receptor-mediated physiological conditions comprising administering to a subject an effective amount of at least one extract selected from the group consisting of:
a hexane extract of Astragalus Membranaceus (HQ); a dichloromethane extract of Astragalus Membranaceus (HQ); a chloroform extract of Astragalus Membranaceus (HQ); and a non-aqueous extract of Astragalus Membranaceus provided that the HQ extract is not an ethanol or butanol HQ extract.

13. The method of claim 12, wherein comprising treating the subject with 5 to 5000 mg of extract for the treatment of PPARα-mediated physiological conditions.

14. The method of claim 12, wherein comprising treating the subject with 5 to 5000 mg of extract for the treatment of PPARγ-mediated physiological conditions.

15. The method of claim 12, wherein comprising treating the subject with 1 to 1000 μg of extract per mL of blood volume for the treatment of PPARα-mediated physiological conditions.

16. The method of claim 12, wherein comprising treating the subject with 1 to 1000 μg of, extract per mL of blood volume for the treatment of PPARγ-mediated physiological conditions.

17. The method of claim 12, wherein the steroid/nuclear (s/n) receptor-mediated physiological conditions is PPAR-mediated physiological condition and is diabetes, cancer, polycystic ovarian disease, or inflammatory bowel disease.

18. The method of claim 12, wherein the steroid/nuclear (s/n) receptor-mediated physiological condition is PPAR-mediated physiological conditions and are dyslipidaemia, atherosclerosis, coronary heart disease or obesity.

19. A method for the treatment PPAR-mediated physiological conditions comprising administering to a subject an effective amount of calycosin and/or formononetin, provided that the treatment of cancer of prostate and breast is excluded.

20. The method of claim 19, wherein the PPAR-mediated physiological condition and is diabetes, atherosclerosis, inflammatory bowel disease, polycystic ovarian disease, obesity, dyslipidaemia and/or the cancer of colon.

21. A method for augmenting or synergising the activity of ligands of steroid/nuclear receptors comprising administering to a subject an effective amount of at least one of:
an extract of Astragalus Membranaceus (HQ);
a flavonoid compound; and
a pharmaceutical or dietary supplement composition comprising an effective amount of an extract of Astragalus Membranaceus or a flavonoid compound;
in the presence of at least one ligand of steroid/nuclear receptors.

22. The method of claim 21, wherein the pharmaceutical or dietary supplement composition comprises an effective amount of at least one extract selected from the group consisting of: a hexane extract of Astragalus Membranaceus (HQ); a dichlorornethane extract of Astragalus Membranaceus (HQ); a chloroform extract of Astragalus Membranaceus (HQ);
and a non-aqueous extract of Astragalus Membranaceus (HQ) provided that the HQ extract is not an ethanol or butanol HQ extract.

23. The method of claim 21, wherein the ligand is a PPAR ligand.

24. The method of claim 21, wherein the ligand is an anti-diabetic or hypolipidaemic drug.

25. The method of claim 21, wherein the ligand is a hormone.

26. The method of claim 25, wherein the hormone is androgen, progestogen or glucocorticoid.

27. The method of claim 21, wherein:
a) the ligand is androgen and the subject is or is not under disease or condition of male infertility, chronic bone and muscle mass loss, geriatric andropause, androgen insensitivity syndromes, Klinefelter syndrome or cryptorchidism;
b) the ligand is progestogen and the subject is or is not under disease or condition of postmenopausal hormone replacement therapy, female infertility, endometrial cancer, secondary amenorrhea, functional uterine bleeding or menstrual disorders; or
c) the ligand is glucocorticoid and the subject is or is not under disease or condition of autoimmune diseases, arthritis, post-operative graft rejection or asthma.

28. The method according to claim 21, wherein the flavonoid compound is calycosin, formononetin, genistein or daidzein.

29. The method according to claim 21, wherein the method is for the treatment of PPARγ-mediated conditions selected from the group consisting of: diabetes, atherosclerosis, polycystic ovarian disease, hormone-dependent cancer of the breast, colon or prostate, of inflammatory bowel disease.

30. The extract of claim 1, which prepared by treating Astragalus Membranaceus plant or part of the plant with hexane, dichloromethane and/or chloroform.

31. A method for screening and/or discovering compounds or extracts capable of augmenting or synergising the activity of ligands of steroid/nuclear receptors comprising;
contacting or mixing a compound or extract to a composition comprising at least one ligand of steroid/nuclear receptors, and
determining the augment or synergism of the activity of the ligand.

32. The method of claim 31, wherein the ligand is bound to a pocket of the steroid/nuclear receptors and the compound or extract does not bind specifically to the ligand-binding pocket of the steroid/nuclear receptor.

33. The method of claim 31, wherein the ligand is an endogenous androgen, progestogen, glucocorticoid and/or a PPAR agonist.
